SEQUENCE LISTING (7 sequences)

SEQ ID NO: 1 - DNA, 4080 bp, Artificial sequence
  Synthetic
gaattcctgcagctcagcagccgccgccagagcaggacgaaccgccaatcgcaaggcacccttaaggacgtcgagtcgtcggcggcggtctcgtcctgcttggcggttagcgttccgtggtctgagaacttcaggatgcagatgtctccagccctcacctgcctagtcctgggcctggccagactcttgaagtcctacgtctacagaggtcgggagtggacggatcaggacccggaccggcttgtctttggtgaagggtctgctgtgcaccatcccccatcctacgtggcccacctggccgaacagaaaccacttcccagacgacacgtggtagggggtaggatgcaccgggtggaccggtcagacttcggggtgagggtgtttcagcaggtggcgcaggcctccaaggaccgcaacgtgagtctgaagccccactcccacaaagtcgtccaccgcgtccggaggttcctggcgttgcacgttttctcaccctatggggtggcctcggtgttggccatgctccagctgacaacaggaggacaaaagagtgggataccccaccggagccacaaccggtacgaggtcgactgttgtcctcctgaaacccagcagcagattcaagcagctatgggattcaagattgatgacaagggcatggccctttgggtcgtcgtctaagttcgtcgataccctaagttctaactactgttcccgtaccggcccgccctccggcatctgtacaaggagctcatggggccatggaacaaggatgagatcagcgggcgggaggccgtagacatgttcctcgagtaccccggtaccttgttcctactctagtcgaccacagacgcgatcttcgtccagcgggatctgaagctggtccagggcttcatgccccactggtgtctgcgctagaagcaggtcgccctagacttcgaccaggtcccgaagtacggggtgttcttcaggctgttccggagcacggtcaagcaagtggacttttcagaggtggagagagccaagaagtccgacaaggcctcgtgccagttcgttcacctgaaaagtctccacctctctcggagattcatcatcaatgactgggtgaagacacacacaaaaggtatgatcagcaacttgctttctaagtagtagttactgacccacttctgtgtgtgttttccatactagtcgttgaacgaagggaaaggagccgtggaccagctgacacggctggtgctggtgaatgccctctacttcaacccctttcctcggcacctggtcgactgtgccgaccacgaccacttacgggagatgaagttgggccagtggaagactcccttccccgactccagcacccaccgccgcctcttccacaaatcaccggtcaccttctgagggaaggggctgaggtcgtgggtggcggcggagaaggtgtttagtgacggcagcactgtctctgtgcccatgatggctcagaccaacaagttcaactatactgagctgccgtcgtgacagagacacgggtactaccgagtctggttgttcaagttgatatgactcttcaccacgcccgatggccattactacgacatcctggaactgccctaccacggggacaccaagtggtgcgggctaccggtaatgatgctgtaggaccttgacgggatggtgcccctgtggctcagcatgttcattgctgccccttatgaaaaagaggtgcctctctctgccctcaccaacgagtcgtacaagtaacgacggggaatactttttctccacggagagagacgggagtggttgattctgagtgcccagctcatcagccactggaaaggcaacatgaccaggctgccccgcctctaagactcacgggtcgagtagtcggtgacctttccgttgtactggtccgacggggcggagctggttctgcccaagttctccctggagactgaagtcgacctcaggaagcccctagagaacgaccaagacgggttcaagagggacctctgacttcagctggagtccttcggggatctcttgctgggaatgaccgacatgttcagacagtttcaggctgacttcacgagtctttcagaccaagacccttactggctgtacaagtctgtcaaagtccgactgaagtgctcagaaagtctggttgagcctctccacgtcgcgcaggcgctgcagaaagtgaagatcgaggtgaacgagagtggcctcggagaggtgcagcgcgtccgcgacgtctttcacttctagctccacttgctctcaccgacggtggcctcctcatccacagctgtcatagtctcagcccgcatggcccccgaggagatctgccaccggaggagtaggtgtcgacagtatcagagtcgggcgtaccgggggctcctctagatcatggacagacccttcctctttgtggtccggcacaaccccacaggaacagtccttttctagtacctgtctgggaaggagaaacaccaggccgtgttggggtgtccttgtcaggaaaagatgggccaagtgatggaaccctgaccctggggaaagacgccttcatctgggacaaaactgtacccggttcactaccttgggactgggacccctttctgcggaagtagaccctgttttgacgagatgcatcgggaaagaagaaactccgaagaaaagaattttagtgttaatgactctttcctctacgtagccctttcttctttgaggcttcttttcttaaaatcacaattactgagaaagtgaaggaagagaagacatttgccttttgttaaaagatggtaaaccagatctgtctccaagacttccttctcttctgtaaacggaaaacaattttctaccatttggtctagacagaggttcaccttggcctctccttggaggacctttaggtcaaactccctagtctccacctgagacccttggaaccggagaggaacctcctggaaatccagtttgagggatcagaggtggactctgggagggagagaagtttgaagcacaactcccttaaggtctccaaaccagacggtgacgcctgcgccctctcttcaaacttcgtgttgagggaattccagaggtttggtctgccactgcggacgcggaccatctggggcacctgcttccacccgtctctctgcccactcgggtctgcagacctggcctggtagaccccgtggacgaaggtgggcagagagacgggtgagcccagacgtctggaccttcccactgaggccctttgcaggatggaactacggggcttacaggagcttttgtgtgcctaagggtgactccgggaaacgtcctaccttgatgccccgaatgtcctcgaaaacacacggaggtagaaactatttctgttccagtcacattgccatcactcttgtactgcctgccaccgcgccatctttgataaagacaaggtcagtgtaacggtagtgagaacatgacggacggtggcgcgaggaggctggtgacaggccaaaggccagtggaagaaacaccctttcatctcagagtccactcctccgaccactgtccggtttccggtcaccttctttgtgggaaagtagagtctcaggtctgtggcactggccacccctccccagtacaggggtgctgcaggtggcagagtgaatgtccgacaccgtgaccggtggggaggggtcatgtccccacgacgtccaccgtctcacttacaggcccatcatgtggcccaactctcctggcctggccatctccctccccagaaacagtgtgcatgggtagtacaccgggttgagaggaccggaccggtagagggaggggtctttgtcacacgtagggttattttggagtgtaggtgacttgtttactcattgaagcagatttctgcttccttttcccaataaaacctcacatccactgaacaaatgagtaacttcgtctaaagacgaaggaaaaatttttataggaatagaggaagaaatgtcagatgcgtgcccagctcttcaccccccaatctaaaaatatccttatctccttctttacagtctacgcacgggtcgagaagtggggggttag

SEQ ID NO: 2 - protein, 402 aa, Artificial sequence [sequence omitted]

SEQ ID NO: 3 - protein, 379 aa, Artificial sequence [sequence omitted]

SEQ ID NO: 4 - DNA, 2352 bp, Artificial sequence
  Synthetic
gtgcaccatcccccatcctacgtggcccacctggcccacgtggtagggggtaggatgcaccgggtggaccggtcagacttcggggtgagggtgtttcagcaggtggcgcaggcctccaaggaccgcaacgtgagtctgaagccccactcccacaaagtcgtccaccgcgtccggaggttcctggcgttgcacgttttctcaccctatggggtggcctcggtgttggccatgctccagctgacaacaggaggacaaaagagtgggataccccaccggagccacaaccggtacgaggtcgactgttgtcctcctgaaacccagcagcagattcaagcagctatgggattcaagattgatgacgcgggcatggccctttgggtcgtcgtctaagttcgtcgataccctaagttctaactactgcgcccgtaccggcccgccctccggcatctgtaccgggagctcatggggccatggaacgcggatgagctcagcgggcgggaggccgtagacatggccctcgagtaccccggtaccttgcgcctactcgagtcgaccacagacgcgatcttcgtccaggcggatctgaagctggtccagggcttcatgccccactggtgtctgcgctagaagcaggtccgcctagacttcgaccaggtcccgaagtacggggtgttcttcaggctgttccggagcacggtcgcgaaagtggacttttcagaggtggagagagccaagaagtccgacaaggcctcgtgccagcgctttcacctgaaaagtctccacctctctcggagattcatcatcaatgactgggtgaagacacacacaaaaggtatgatcagcaacttgctttctaagtagtagttactgacccacttctgtgtgtgttttccatactagtcgttgaacgaagggaaaggagccgtggaccagctgacacggctggtgctggtgaatgccctctacttcaacccctttcctcggcacctggtcgactgtgccgaccacgaccacttacgggagatgaagttgggccagtgggcgactcccttccccgactccagcacccaccgccgcctcttccacaaatcaccggtcacccgctgagggaaggggctgaggtcgtgggtggcggcggagaaggtgtttagtgacggcagcactgtctctgtgcccatgatggctcagaccaacgcgttcaactatactgagctgccgtcgtgacagagacacgggtactaccgagtctggttgcgcaagttgatatgactcttcaccacgcccgatggccattactacgacatcctggaactgccctaccacggggacaccaagtggtgcgggctaccggtaatgatgctgtaggaccttgacgggatggtgcccctgtggctcagcatgttcattgctgccccttatgaaaaagaggtgcctctctctgccctcaccaacgagtcgtacaagtaacgacggggaatactttttctccacggagagagacgggagtggttgattctgagtgcccagctcatcagccactgggcaggcaacatgaccaggctgccccgcctctaagactcacgggtcgagtagtcggtgacccgtccgttgtactggtccgacggggcggagctggttctgcccaagttctccctggagactgaagtcgacctcaggaagcccctagagaacgaccaagacgggttcaagagggacctctgacttcagctggagtccttcggggatctcttgctgggaatgaccgacatgttcagacagtttcaggctgacttcacgagtctttcagaccaagacccttactggctgtacaagtctgtcaaagtccgactgaagtgctcagaaagtctggttgagcctctccacgtcgcgcaggcgctgcagaaagtgaagatcgaggtgaacgagagtggcctcggagaggtgcagcgcgtccgcgacgtctttcacttctagctccacttgctctcaccgacggtggcctcctcatccacagctgtcatagcctcagccgtcatggcccccgaggagatctgccaccggaggagtaggtgtcgacagtatcggagtcggcagtaccgggggctcctctagatcatggacagacccttcctctttgtggtccggcacaaccccacaggaacagtccttttctagtacctgtctgggaaggagaaacaccaggccgtgttggggtgtccttgtcaggaaaagatgggccaagtgatggaaccctgaccctggggaaagacgccttcatctgggacaaaactgtacccggttcactaccttgggactgggacccctttctgcggaagtagaccctgttttgac

SEQ ID NO: 5 - protein, 379 aa, Artificial sequence
  Synthetic
VHHPPSYVAHLASDFGVRVFQQVAQASKDRNVVFSPYGVASVLAMLQLTTGGETQQQIQAAMGFKIDDAGMAPALRHLYAELMGPWNADELSTTDAIFVQADLKLVQGFMPHFFRLFRSTVAKVDFSEVERARFIINDWVKTHTKGMISNLLGKGAVDQLTRLVLVNALYFNGQWATPFPDSSTHRRLFHKSDGSTVSVPMMAQTNAFNYTEFTTPDGHYYDILELPYHGDTLSMFIAAPYEKEVPLSALTNILSAQLISHWAGNMTRLPRLLVLPKFSLETEVDLRKPLENLGMTDMFRQFQADFTSLSDQEPLHVAQALQKVKIEVNESGTVASSSTAVIASAVMAPEEIIMDRPFLFVVRHNPTGTVLFMGQVMEP

SEQ ID NO: 6 - DNA, 3834 bp, Artificial sequence
  Synthetic
atgtacaggatgcaactcctgtcttgcattgcactaagtcttgcacttgcacttgtcacgtacatgtcctacgttgaggacagaacgtaacgtgattcagaacgtgaacgtgaacagtgcaattcggcacctctcgagcccaaatctagtgacaaaactcacacatgcccaccgtgcccattaagccgtggagagctcgggtttagatcactgttttgagtgtgtacgggtggcacgggtgcacctgaactcctggggggaccgtcagtcttcctcttccccccaaaacccaaggacacccgtggacttgaggacccccctggcagtcagaaggagaaggggggttttgggttcctgtggctctacatcacccgggaacctgaggtcacatgcgtggtggtggacgtgagccacgaagacgagatgtagtgggcccttggactccagtgtacgcaccaccacctgcactcggtgcttctgcctgaggtcaagttcaactggtacgtggacggcgtggaggtgcataatgccaagacaaagggactccagttcaagttgaccatgcacctgccgcacctccacgtattacggttctgtttcccgcgggaggagcagtacaacagcacgtaccgtgtggtcagcgtcctcaccgtcctgcacggcgccctcctcgtcatgttgtcgtgcatggcacaccagtcgcaggagtggcaggacgtgcaggactggctgaatggcaaggagtacaagtgcaaggtctccaacaaagccctcccagccgtcctgaccgacttaccgttcctcatgttcacgttccagaggttgtttcgggagggtcggcccatcgagaaaaccatctccaaagccaaagggcagccccgagaaccacaggtgtacaccgggtagctcttttggtagaggtttcggtttcccgtcggggctcttggtgtccacatgtggctgcccccatcccgggaggagatgaccaagaaccaggtcagcctgacctgcctggtcaaagacgggggtagggccctcctctactggttcttggtccagtcggactggacggaccagtttggcttctatcccagcgacatcgccgtggagtgggagagcaatgggcagccggagaacaacccgaagatagggtcgctgtagcggcacctcaccctctcgttacccgtcggcctcttgttgtacaagaccacgcctcccgtgctggactccgacggctccttcttcctctacagcaagctcatgttctggtgcggagggcacgacctgaggctgccgaggaagaaggagatgtcgttcgagaccgtggacaagagcaggtggcagcaggggaacgtcttctcatgctccgtgatgcatgagtggcacctgttctcgtccaccgtcgtccccttgcagaagagtacgaggcactacgtactcgctctgaagttccactacacgcagaagagcctctccctgtctccgggtgcaactagtgtgcgagacttcaaggtgatgtgcgtcttctcggagagggacagaggcccacgttgatcacaccaccatcccccatcctacgtggcccacctggcctcagacttcggggtgagggtgtttcaggtggtagggggtaggatgcaccgggtggaccggagtctgaagccccactcccacaaagtccaggtggcgcaggcctccaaggaccgcaacgtggttttctcaccctatggggtggcctcggtccaccgcgtccggaggttcctggcgttgcaccaaaagagtgggataccccaccggagcgtgttggccatgctccagctgacaacaggaggagaaacccagcagcagattcaagcagctcacaaccggtacgaggtcgactgttgtcctcctctttgggtcgtcgtctaagttcgtcgaatgggattcaagattgatgacaagggcatggcccccgccctccggcatctgtacaaggagtaccctaagttctaactactgttcccgtaccgggggcgggaggccgtagacatgttcctcctcatggggccatggaacaaggatgagctcagcaccacagacgcgatcttcgtccaggcggagtaccccggtaccttgttcctactcgagtcgtggtgtctgcgctagaagcaggtccgcgatctgaagctggtccagggcttcatgccccacttcttcaggctgttccggagcacggtcctagacttcgaccaggtcccgaagtacggggtgaagaagtccgacaaggcctcgtgccagaagaaagtggacttttcagaggtggagagagccagattcatcatcaatgactgggtgaagttctttcacctgaaaagtctccacctctctcggtctaagtagtagttactgacccacttcacacacacaaaaggtatgatcagcaacttgcttgggaaaggagccgtggaccagctgacatgtgtgtgttttccatactagtcgttgaacgaaccctttcctcggcacctggtcgactgtcggctggtgctggtgaatgccctctacttcaacggccagtggaagactcccttccccgacgccgaccacgaccacttacgggagatgaagttgccggtcaccttctgagggaaggggctgtccagcacccaccgccgcctcttccacaaatcagacggcagcactgtctctgtgcccatgaggtcgtgggtggcggcggagaaggtgtttagtctgccgtcgtgacagagacacgggtacatggctcagaccaacaagttcaactatactgagttcaccacgcccgatggccattactactaccgagtctggttgttcaagttgatatgactcaagtggtgcgggctaccggtaatgatggacatcctggaactgccctaccacggggacaccctcagcatgttcattgctgccccttatctgtaggaccttgacgggatggtgcccctgtgggagtcgtacaagtaacgacggggaatagaaaaagaggtgcctctctctgccctcaccaacattctgagtgcccagctcatcagccacctttttctccacggagagagacgggagtggttgtaagactcacgggtcgagtagtcggtgtggaaaggcaacatgaccaggctgccccgcctcctggttctgcccaagttctccctggagacctttccgttgtactggtccgacggggcggaggaccaagacgggttcaagagggacctcactgaagtcgacctcaggaagcccctagagaacctgggaatgaccgacatgttcagacagtgacttcagctggagtccttcggggatctcttggacccttactggctgtacaagtctgtctttcaggctgacttcacgagtctttcagaccaagagcctctccacgtcgcgcaggcgctgaaagtccgactgaagtgctcagaaagtctggttctcggagaggtgcagcgcgtccgcgaccagaaagtgaagatcgaggtgaacgagagtggcacggtggcctcctcatccacagctgtcgtctttcacttctagctccacttgctctcaccgtgccaccggaggagtaggtgtcgacagatagcctcagccgtcatggcccccgaggagatcatcatggacagacccttcctctttgtgtatcggagtcggcagtaccgggggctcctctagtagtacctgtctgggaaggagaaacacgtccggcacaaccccacaggaacagtccttttcatgggccaagtgatggaaccctgacaggccgtgttggggtgtccttgtcaggaaaagtacccggttcactaccttgggact

SEQ ID NO: 7 - protein, 638 aa, Artificial sequence
  Synthetic
MYRMQLLSCIALSLALALVTNSAPLEPKSSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALKFHYTQKSLSLSPGATSVHHPPSYVAHLASDFGVRVFQQVAQASKDRNVVFSPYGVASVLAMLQLTTGGETQQQIQAAMGFKIDDKGMAPALRHLYKELMGPWNKDELSTTDAIFVQADLKLVQGFMPHFFRLFRSTVKKVDFSEVERARFIINDWVKTHTKGMISNLLGKGAVDQLTRLVLVNALYFNGQWKTPFPDSSTHRRLFHKSDGSTVSVPMMAQTNKFNYTEFTTPDGHYYDILELPYHGDTLSMFIAAPYEKEVPLSALTNILSAQLISHWKGNMTRLPRLLVLPKFSLETEVDLRKPLENLGMTDMFRQFQADFTSLSDQEPLHVAQALQKVKIEVNESGTVASSSTAVIASAVMAPEEIIMDRPFLFVVRHNPTGTVLFMGQVMEP

(19) United States
(12) Patent Application Publication
(10) Pub. No.: US 2022/0372111 A1
(43) Pub. Date: Nov. 24, 2022

(54) POLYPEPTIDE INHIBITORS OF NEUTROPHIL ELASTASE ACTIVITY AND USES THEREOF

(71) Applicant: The Regents of the University of Michigan, Ann Arbor, MI (US)

(72) Inventor: Daniel Lawrence, Ann Arbor, MI (US)

(21) Appl. No.: 17/778,306

(22) PCT Filed: Nov. 19, 2020
(86) PCT No.: PCT/US2020/061347

§ 371 Date: May 19, 2022

Related U.S. Application Data
(60) Provisional application No. 62/938,859, filed on Nov. 21, 2019.

(51) Int. Cl. C07K 14/81; A61P 11/00; A61K 48/00

(52) U.S. Cl. CPC C07K 14/8132; A61P 11/00; A61K 48/005; C07K 2319/30; A61K 38/00

(57) ABSTRACT

The invention features polypeptides that include variants of plasminogen activator inhibitor 1 (PAI-1) having a reduced ability to bind with vitronectin, having a reduced ability to interact with the PAI-1 clearance receptor LDL receptor-related protein 1 (LRP1), and having the ability to efficiently inhibit neutrophil elastase in the presence of neutrophil extracellular traps (NETS). In some embodiments, a polypeptide of the invention includes PAI-1 variants optionally fused to an Fc domain monomer or moiety. The invention also features pharmaceutical compositions and methods of using the polypeptides to treat diseases and conditions characterized with aberrant neutrophil elastase activity (e.g., Idiopathic Pulmonary Fibrosis).

### CROSS REFERENCE TO RELATED APPLICATION

This application claims priority to and the benefit of U.S. Provisional Application No. 62/938,859, filed Nov. 21, 2019, which is hereby incorporated by reference in its entirety.

### FIELD OF THE INVENTION

The invention features polypeptides that include variants of plasminogen activator inhibitor 1 (PAI-1) having a reduced ability to bind with vitronectin, having a reduced ability to interact with the PAI-1 clearance receptor LDL receptor-related protein 1 (LRP1), and having the ability to efficiently inhibit neutrophil elastase (NE) in the presence of neutrophil extracellular traps (NETs). In some embodiments, a polypeptide of the invention includes PAI-1 variants optionally fused to an Fc domain monomer or moiety. The invention also features pharmaceutical compositions and methods of using the polypeptides to treat diseases and conditions characterized with aberrant neutrophil elastase activity (e.g., Idiopathic Pulmonary Fibrosis).

### INTRODUCTION

Idiopathic Pulmonary Fibrosis (IPF) is a progressive and chronic lung disease that results in respiratory failure and death. IPF is the most common cause of death from progressive lung disease, and worldwide effects about 5 million people. Estimated median survival after diagnosis is only 3-5 years (see, Chakraborty et al., (2014) Expert Opin Investig Drugs, 23:893-910; Spagnolo et al., (2015) Pharmacology & Therapeutics 152:18-27; Tzouvelekis et al., (2015) Therapeutics and Clinical Risk Management 11:359-370; Lederer D J and Martinez F J. The New England journal of medicine. 2018; 378:1811-1823). There are approximately 130,000 IPF patients in the US with an estimated 30,000 to 40,000 new cases diagnosed annually (see, Ley, B., and Collard, H. R. 2013. Epidemiology of idiopathic pulmonary fibrosis. Clin. Epidemiol. 5:483-492; Lynch, J. P., III, et al., 2016. Idiopathic Pulmonary Fibrosis: Epidemiology, Clinical Features, Prognosis, and Management. Semin. Respir. Crit Care Med. 37:331-357). Prevalence of IPF ranges from 14.0 to 42.7 cases per 100,000 persons and the annual incidence ranges from 6.8 to 16.3 cases per 100,000 persons, depending on the strictness of the diagnostic criteria employed (see, Jones, M. G., and Richeldi, L. Semin. Respir. Crit. Care Med. 2016; 37:477-484). The prevalence of IPF increases with age, with most IPF patients at the age of 60 years or even older at the time of diagnosis. The disease is more common in men than in women (see, Fernandez Perez E R et al., (2010) Chest 137(1):129-137), with most patients being current or former smokers. (see, Jones, M G and Richeldi, L., Semin. Respir. Care Med. 2016, 37:477-484).

The etiology of IPF remains unknown. Potential factors, such as cigarette smoking, dust exposure and infection agents, however, have been associated with the development of IPF. IPF is characterized by progressive and irreversible distortion of the lung's architecture as a result of apoptosis of epithelial and endothelial cells, fibroblast hyperplasia and extracellular matrix remodeling (see, Chakraborty et al., (2014) Expert Opin Investig Drugs, 23:893-910). As interstitial fibrosis advances with accompanying distortion of lung architecture, the lung becomes less compliant, increasing the effort associated with breathing, leading to dyspnea. Typically, lung function declines slowly over time, but some patients experience rapid declines that can lead to hospitalization or death, particularly in later stages of the disease.

Development of agents for treatment of IPF has been slow in progress. The first two agents for treating IPF, pirfenidone and nintedanib, were approved only at the end of 2014 (see, King et al., (2014) N Engl J Med 370:2083-92; Richeldi et al., (2014) N Engl J Med 370:2071-82; Richeldi, L, et al., Am. J. Med. Sci. 2019, 357:370-373). These two agents, however, have only limited efficacy and significant side effects, and require complicated dosing regimen. Recently conducted phase 3 clinical trials of pirfenidone, sildenafil, bosentan, etanercept, and interferon gamma-1b failed to demonstrate efficacy in their primary endpoints. N-acetyl cysteine (NAC), corticosteroids, and the immunosuppressive drugs cyclophosphamide and azathioprine are commonly prescribed, but there is little evidence that use of these drugs improves patient outcome or alters the natural course of the disease (see, Collard H R et al., (2004) Chest 125(6):2169-2174; Walter N et al., (2006) Proc Am Thorac Soc 3(4):377-381). Lung transplantation is the only treatment that improves survival, but most IPF patients are not eligible for transplantation because of their age or comorbid conditions. IPF patients usually are managed with supportive measures such as symptomatic treatment of cough and dyspnea, supplemental oxygen for hypoxemia, smoking cessation, pulmonary rehabilitation, and prophylaxis and control of respiratory tract infections.

Improved methods for treating IPF are needed.

The present invention addresses this need.

### SUMMARY OF THE INVENTION

While it is well established that complexes of plasminogen-activator inhibitor 1 (PAI-1) with target enzymes bind tightly to LDL receptor-related protein 1 (LRP1), the molecular details of this interaction are not well defined. Further, there is considerable controversy in the literature regarding the nature of the interaction of free PAI-1 with LRP1. In experiments conducted during the course of developing embodiments for the present invention the binding of free PAI-1 and complexes of PAI-1 with low molecular-weight urokinase-type plasminogen activator (uPA) to LRP1 were examined. The data confirm that uPA:PAI-1 complexes bind to LRP1 with ˜100-fold increased affinity over PAI-1 alone. Chemical modification of PAI-1 confirms an essential requirement of lysine residues on PAI-1 for the interactions of both PAI-1 and uPA:PAI-1 complexes to LRP1. Surface plasmon resonance measurements support a bivalent binding model in which multiple sites on PAI-1 and uPA:PAI-1 complexes interact with complementary sites on LRP1. The ionic strength dependence of binding suggest the involvement of 2 critical charged residues for the interaction of PAI-1 with LRP1 and 3 charged residues for the interaction of uPA:PAI-1 complexes with LRP1. The enhanced affinity resulting from the interaction of three regions of the uPA:PAI-1 complex with LDLa repeats on LRP1 provides a molecular explanation for the increased affinity of uPA:PAI-1 complexes for LRP1. Mutational analysis reveals overlap between LRP1 binding and the binding site for a small molecule inhibitor of PAI-1, CDE-096 (a specific PAI-1 inhibitor), with an important role for K207 in the interaction of PAI-1 with LRP1 and K207, K88 and K80 for the interaction of uPA:PAI-1 complexes with LRP1.

Experiments conducted during the course of developing embodiments of the present invention clarified the relative binding affinities of PAI-1 and protease:PAI-1 complexes with LRP1. In addition, experiments were conducted to determine if the binding of a protease:PAI-1 complex to LRP1 is mainly attributed to determinants on PAI-1. In addition, experiments were conducted to determine the specific amino acid residues in free PAI-1 and PAI-1 in complex with a target protease (urokinase-type plasminogen activator (uPA)) that participate in their binding to LRP1. Indeed, mutational analysis revealed overlap between LRP1 binding and the binding site for a small molecule inhibitor of PAI-1, CDE-096, with an important role for K207 in the interaction of PAI-1 with LRP1 and K207, K88 and K80 for the interaction of uPA:PAI-1 complexes with LRP1.

IPF is characterized by interstitial scar tissue formation that can dramatically restrict lung function. There are approximately 130,000 IPF patients in the US with an estimated 30,000 to 40,000 new cases diagnosed annually (see, Ley, B., and Collard, H. R. 2013. Epidemiology of idiopathic pulmonary fibrosis. Clin. Epidemiol. 5:483-492; Lynch, J. P., III, et al., 2016. Idiopathic Pulmonary Fibrosis: Epidemiology, Clinical Features, Prognosis, and Management. Semin. Respir. Crit Care Med. 37:331-357). Life expectancy following a diagnosis of IPF is generally three to five years (see, Lederer, D. J. and Martinez, F. J., NEJM 2018, 378:1811-1823). There is no effective therapeutic treatment except lung transplant. The lack of therapies that halt the progression of IPF presents a significant challenge and is a major unmet medical need. The two currently approved drugs, pirfenidone and nintedanib, have been shown to slow disease progression but they do not stop progression and cannot restore lost lung function. As such, a novel therapeutic for IPF treatment is necessary for disease management.

The present invention addresses this need through providing compositions comprising mutant PA1-I polypeptides capable of inhibiting NE, and in particular, inhibiting NE bound in NETs. Provided herein are PAI-1 variants capable of inhibiting NE activity while having a diminished ability to bind with vitronectin and LRP1. Indeed, experiments conducted during the course of developing embodiments for the present invention demonstrated such PAI-1 variants to have improved efficacy for treating conditions associated with NE activity (e.g., IPF) through inhibiting its ability to bind with vitronectin through modifying the PAI-1 amino acid residues responsible for such vitronectin binding (e.g., R101A and Q123K).

Indeed, experiments conducted during the course of developing embodiments for the present invention utilized a serpin mapping technology and identified mutant forms of PA1-1 (e.g., having one or more of the following mutations within wild-type human mature PAI-1 amino acid sequence (SEQ ID NO: 3): K69A, K80A, K88A, I91L, R101A, K122A, Q123K, K176A, K207A, K263A, V343A, and R346V) as an inhibitor of NE capable of efficient inhibition of NE within the NETs environment. Such mutant forms of PA1-I were shown to irreversibly inhibit NE bound to DNA, a major component of NETs, where the FDA approved human plasma derived A1AT trademarked Aralast is ineffective. In certain embodiments, such mutant forms of PAI-1 are further associated with the human IgG-Fc.

Accordingly, the present invention features polypeptides that include PAI-1 variants capable of inhibiting NE activity while having a diminished ability to bind with vitronectin through modifying its amino acid residues responsible for vitronectin binding (e.g., R101A and Q123K), and resulting in improved pharmacokinetics (PK) through modifying its amino acid residues responsible for LRP1 binding (e.g., K207, K88 and K80) resulting in improved efficacy for treating conditions associated with NE activity (e.g., IPF). In some embodiments, a polypeptide of the invention includes a PAI-1 variant fused to the N- or C-terminus of an Fc domain monomer or moiety (e.g., for purposes of improving PK). In some embodiments, a polypeptide of the invention includes a PAI-1 variant fused to the N- or C-terminus of an Fc domain monomer or moiety. In some embodiments, the Fc domain monomer or moiety increases stability or improves the pharmacokinetics of the polypeptide.

Such moieties may be fused or attached by amino acid or other covalent bonds and may increase stability of the polypeptide. A polypeptide including a PAI-1 variant fused to an Fc domain monomer may also form a dimer (e.g., a homodimer or heterodimer) through the interaction between two Fc domain monomers. In some embodiments, a polypeptide described herein attached with an Fc domain monomer is fused to the polypeptide by way of a linker. In some embodiments, the linker is an amino acid spacer.

The polypeptides of the invention may be used to inhibit NE activity and may be used to inhibit NE activity bound in NETs. In addition, the polypeptides of the invention may be used to treat a subject having a condition characterized with aberrant NE activity (e.g., IPF). In addition, the polypeptides of the invention may be used to prevent a subject from infliction of a condition characterized with aberrant NE activity (e.g., IPF). Further, the polypeptides of the invention may also be used to affect NE activity in a subject having a risk of developing or having a disease or condition involving aberrant NE activity.

In some embodiments, the invention features a polypeptide including PAI-1 variant, the variant having one or more of the following mutations within wild-type human mature PAI-1 amino acid sequence (SEQ ID NO: 3): K69A, K80A, K88A, I91L, R101A, K122A, Q123K, K176A, K207A, K263A, V343A, and R346V. In some embodiments, the PAI-1 variant includes mutations within wild-type human mature PAI-1 amino acid sequence (SEQ ID NO: 3): K69A, K80A, K88A, I91L, R101A, K122A, Q123K, K176A, K207A, K263A, V343A, and R346V as shown in SEQ ID NO: 5. In some embodiments, the PAI-1 variant includes the following mutations within wild-type human mature PAI-1 amino acid sequence (SEQ ID NO: 3): R101A and Q123K. In some embodiments, the PAI-1 variant includes the following mutations within wild-type human mature PAI-1 amino acid sequence (SEQ ID NO: 3): I91L, R101A, Q123K, V343A, R346V. In some embodiments, the PAI-1 variant includes the following mutations within wild-type human mature PAI-1 amino acid sequence (SEQ ID NO: 3): I91L, R101A, Q123K, K207A, V343A, R346V. This mutant should have a prolonged half-life, especially in the Fc-form, with full activity against NE in NETS.

In some embodiments, the invention features a polypeptide including PAI-1 variant attached with an Fc domain monomer or moiety, the variant having one or more of the following mutations within wild-type human mature PAI-1 amino acid sequence (SEQ ID NO: 3): K69A, K80A, K88A, I91L, R101A, K122A, Q123K, K176A, K207A, K263A, V343A, and R346V. In some embodiments, the PAI-1 variant attached with an Fc domain monomer or moiety includes the following mutations within wild-type human mature PAI-1 amino acid sequence (SEQ ID NO: 3): K69A, K80A, K88A, I91L, R101A, K122A, Q123K, K176A, K207A, K263A, V343A, and R346V. In some embodiments, the PAI-1 variant attached with an Fc domain monomer or moiety includes the following mutations within wild-type human mature PAI-1 amino acid sequence (SEQ ID NO: 3): R101A and Q123K. In some embodiments, the PAI-1 variant attached with an Fc domain monomer or moiety includes the following mutations within wild-type human mature PAI-1 amino acid sequence (SEQ ID NO: 3): I91L, R101A, Q123K, V343A, R346V (SEQ ID NO: 7). In some embodiments, the PAI-1 variant attached with an Fc domain monomer or moiety includes the following mutations within wild-type human mature PAI-1 amino acid sequence (SEQ ID NO: 3): I91L, R101A, Q123K, K207A, V343A, R346V.

In some embodiments, the polypeptide described herein is capable of inhibiting NE, and in particular, inhibiting NE bound in NETs.

In some embodiments, the invention features a nucleic acid molecule encoding a polypeptide described herein (e.g., a polypeptide including a PAI-1 variant having one or more of the following mutations within wild-type human mature PAI-1 amino acid sequence (SEQ ID NO: 3): K69A, K80A, K88A, I91L, R101A, K122A, Q123K, K176A, K207A, K263A, V343A, and R346V. In another aspect, the invention also features a vector including the nucleic acid molecule described herein.

In another aspect, the invention features a host cell that expresses a polypeptide described herein, wherein the host cell includes a nucleic acid molecule or a vector described in the previous two aspects, wherein the nucleic acid molecule or vector is expressed in the host cell.

In another aspect, the invention features a method of preparing a polypeptide described herein, wherein the method includes: a) providing a host cell including a nucleic acid molecule or a vector described herein, and b) expressing the nucleic acid molecule or vector in the host cell under conditions that allow for the formation of the polypeptide.

In another aspect, the invention features a pharmaceutical composition including a polypeptide, nucleic acid molecule, or vector described herein and one or more pharmaceutically acceptable carriers or excipients. In some embodiments of the pharmaceutical composition, the polypeptide, nucleic acid molecule, or vector is in a therapeutically effective amount.

In another aspect, the invention also features a construct including two identical polypeptides (e.g., a homodimer) each including a PAI-1 variant having one or more of the following mutations within wild-type human mature PAI-1 amino acid sequence (SEQ ID NO: 3): K69A, K80A, K88A, I91L, R101A, K122A, Q123K, K176A, K207A, K263A, V343A, and R346V, wherein the variant is fused to the N- or C-terminus of an Fc domain monomer. The two Fc domain monomers in the two polypeptides interact to form an Fc domain in the construct.

In another aspect, the invention also features a construct including two different polypeptides (e.g., a heterodimer) each including a PAI-1 variant having a different combination of one or more of the following mutations within wild-type human mature PAI-1 amino acid sequence (SEQ ID NO: 3): K69A, K80A, K88A, I91L, R101A, K122A, Q123K, K176A, K207A, K263A, V343A, and R346V, wherein the two variants are fused to the N- or C-terminus of an Fc domain monomer. The two Fc domain monomers in the two polypeptides interact to form an Fc domain in the construct.

In another aspect, the invention features a method of inhibiting NE activity in a subject in need thereof. In another aspect, the invention features a method of inhibiting NE activity bound in NETs in a subject in need thereof. The methods include administering to the subject a therapeutically effective amount of a polypeptide, nucleic acid molecule, or vector described herein or a pharmaceutical composition described herein.

In some embodiments of the method of inhibiting NE activity or NE activity bound within NETs in a subject, the subject has IPF and/or a condition characterized with aberrant NE activity (e.g., cystic fibrosis, chronic obstructive pulmonary disease (COPD), emphysema). In some embodiments of the method of inhibiting NE activity or NE activity bound within NETs in a subject, the subject has an A1AT activity and/or expression deficiency.

In another aspect, the invention features a method of treating a subject having IPF by administering to the subject a therapeutically effective amount of a polypeptide, nucleic acid molecule, or vector described herein or a pharmaceutical composition described herein.

In another aspect, the invention features a method of treating a subject having cystic fibrosis by administering to the subject a therapeutically effective amount of a polypeptide, nucleic acid molecule, or vector described herein or a pharmaceutical composition described herein.

In another aspect, the invention features a method of treating a subject having COPD by administering to the subject a therapeutically effective amount of a polypeptide, nucleic acid molecule, or vector described herein or a pharmaceutical composition described herein.

In another aspect, the invention features a method of treating a subject having emphysema by administering to the subject a therapeutically effective amount of a polypeptide, nucleic acid molecule, or vector described herein or a pharmaceutical composition described herein.

In another aspect, the invention features a method of treating a subject having acute respiratory distress syndrome (ARDS) by administering to the subject a therapeutically effective amount of a polypeptide, nucleic acid molecule, or vector described herein or a pharmaceutical composition described herein.

In another aspect, the invention features a method of treating a subject having ischemia reperfusion injury by administering to the subject a therapeutically effective amount of a polypeptide, nucleic acid molecule, or vector described herein or a pharmaceutical composition described herein.

In another aspect, the invention features a method of treating a subject having ethanol induced chronic pancreatitis by administering to the subject a therapeutically effective amount of a polypeptide, nucleic acid molecule, or vector described herein or a pharmaceutical composition described herein.

In another aspect, the invention features a method of treating a subject having rheumatoid arthritis (RA) by administering to the subject a therapeutically effective amount of a polypeptide, nucleic acid molecule, or vector described herein or a pharmaceutical composition described herein.

In another aspect, the invention features a method of treating a subject having disseminated intravascular coagulation (DIC) by administering to the subject a therapeutically effective amount of a polypeptide, nucleic acid molecule, or vector described herein or a pharmaceutical composition described herein.

In another aspect, the invention features a method of treating a subject having ulcerative colitis (UC) by administering to the subject a therapeutically effective amount of a polypeptide, nucleic acid molecule, or vector described herein or a pharmaceutical composition described herein.

In another aspect, the invention features a method of treating a subject having Crohn's disease by administering to the subject a therapeutically effective amount of a polypeptide, nucleic acid molecule, or vector described herein or a pharmaceutical composition described herein.

In another aspect, the invention features a method of treating a subject having dermatological diseases with neutrophil pathology by administering to the subject a therapeutically effective amount of a polypeptide, nucleic acid molecule, or vector described herein or a pharmaceutical composition described herein.

In another aspect, the invention features a method of treating a subject having an A1AT activity and/or expression deficiency by administering to the subject a therapeutically effective amount of a polypeptide, nucleic acid molecule, or vector described herein or a pharmaceutical composition described herein.

In another aspect, the invention features a method of treating a subject having any condition characterized with aberrant NE activity and/or expression by administering to the subject a therapeutically effective amount of a polypeptide, nucleic acid molecule, or vector described herein or a pharmaceutical composition described herein.

In another aspect, the invention features a method of treating a subject having any condition characterized with deficient A1AT activity and/or expression by administering to the subject a therapeutically effective amount of a polypeptide, nucleic acid molecule, or vector described herein or a pharmaceutical composition described herein.

In some embodiments of any of the above aspects, the subject has or is at risk of developing a condition characterized with aberrant NE activity (e.g., IPF, COPD, cystic fibrosis, emphysema, ARDS, ischemia reperfusion, chronic pancreatitis, RA, DIC, UC, Chron's disease, dermatological diseases).

In some embodiments of any of the above aspects, the subject has or is at risk of developing a condition characterized with deficient A1AT activity and/or expression.

### BRIEF DESCRIPTION OF DRAWINGS

FIG. 1. Essential role for lysine residues on PAI-1 for the binding of PAI-1 and LMWuPA:PAI-1 complexes to LRP1. A. Binding of LMWuPA:PAI-1 complexes and free PAI-1 to LRP1 analyzed by SPR in which Req values were determined by equilibrium measurements. Three independent experiments were performed, and the mean±SE are plotted. KD values (0.9±0.2 nM for LMWuPA:PAI-1 and 74±13 nM for PAI-1) were determined by non-linear regression analysis. B. PAI-1 (lane 1) and chemically modified PAI-1 (lane 2) form complexes with LMWuPA (lanes 3 and 4, respectively). Lane 5, LMWuPA. C. 250 nM PAI-1 and 300 nM PAI-1 chemically modified with sulfo-NHS-acetate were injected over an SPR chip to which LRP1 was immobilized. D. 9 nM of LMWuPA:PAI-1 complex and 80 nM of complex formed with chemically modified PAI-1 were injected over an SPR chip to which LRP1 was immobilized.

FIG. 2. Binding of PAI-1 to LRP1 is ionic strength dependent. A. Increasing concentrations of PAI-1 in a buffer containing increasing concentrations of NaCl were injected over LRP-1 coated-SPR chips and Req values determined. The data are normalized to Rmax for each NaCl concentration. The concentration of NaCl from top curve down: 150 mM, 250 mM, 500 mM, 750 mM and 1000 mM. B. Debye-Hückel plot of PAI-1 binding to LRP1. The KD value at each ionic strength (150, 250, 500, 750 and 1000 mM NaCl) was measured by equilibrium SPR measurements. Three independent experiments were performed, and the values plotted are means±SE. A slope of 1.5±0.1 was determined by linear regression analysis. A similar value for the slope was obtained by averaging the results from linear regression analysis of individual experiments.

FIG. 3. Binding of PAI-1 to LRP1 is well described by a bivalent binding model. (A) Schematic of a bivalent binding model for the interaction of two distinct regions on PAI-1 with complementary sites on LRP1. (B) Increasing concentrations of PAI-1 (9.4, 37.5, 75, 300 nM) were injected over the LRP1-coupled SPR chip. The dissociation of each concentration was measured from the SPR data, with the initial value at t=0 normalized to 100%. Data were fit to a two-exponential decay (blue line). C. Increasing concentrations of PAI-1 (3.9, 7.8, 15.6, 31.2, 62.5, 125 nM) were injected over the LRP1-coupled chip. Fits of the experimental data (black lines) to a bivalent binding model are shown as blue lines. The data shown is a representative experiment from six independent experiments that were performed.

FIG. 4. Binding of PAI-1 to cluster IV from LRP1 fits well to a bivalent binding model. (A) Schematic showing domain organization of LRP1. Clusters of ligand binding repeats (red circles) are labeled, I, II, III and IV. (B) Increasing concentrations of PAI-1 (9.4, 15.6, 31, 62.5, 125 nM) were injected over the LRP1 cluster IV-coupled SPR chip. The dissociation of each concentration was measured from the SPR data, with the initial value at t=0 normalized to 100%. Data were fit to a two-exponential decay (blue line). C. Increasing concentrations of PAI-1 (3.9, 7.8, 15.6, 31.2, 62.5, 125 nM) were injected over the LRP1 cluster IV-coupled chip. Fits of the experimental data (black lines) to a bivalent binding model are shown as blue lines. The data shown are representative of three independent experiments that were performed.

FIG. 5. CDE-096 inhibits the binding of HMWuPA:PAI-1 complexes to LRP1. A. 1 nM of HMWuPA:PAI-1 complex was flowed over an LRP1 coupled SPR chip in the absence (top curve) and presence of increasing concentrations of CDE-096 (15.6, 31.2, 62.5, 125, 250, 500 nM). B. Plots of the initial slopes of the association phase from panel A vs CDE-096 concentration. An IC50 of 70±11 nM was determined by non-linear regression analysis. The data are representative of two independent experiments.

FIG. 6. Binding of LMWuPA:PAI-1 complexes to LRP1 is ionic strength dependent. A. Increasing concentrations of uPA:PAI-1 complexes were flowed over LRP-1 coated-SPR chips in the presence of increasing concentrations of NaCl, and Req values determined. The data are normalized to Rmax for each NaCl concentration. NaCl concentrations from top curve down: 150 mM, 250 mM, 500 mM, 750 mM and 1000 mM. B. Debye-Hückel plot of LMWuPA:PAI-1 binding to LRP1. The KD value at each ionic strength (150 mM, 250 mM, 500 mM, 750 mM and 1000 mM NaCl) was measured by equilibrium SPR measurements. Three independent experiments were performed, and the mean±SE are plotted. A slope of 2.4±0.4 was determined by linear regression analysis. An identical value was obtained by averaging the results from linear regression analysis of individual experiments.

FIG. 7. LMWuPA:PAI-1 complexes bind to LRP1 via a complex kinetic model. A) Model used for analyzing the binding of LMWuPA:PAI-1 complexes to LRP1. In Scheme I, LMWuPA:PAI-1 binds via a bivalent model. At higher concentrations of LMWuPA:PAI-1, a monovalent model of binding occurs (Scheme II). B) Increasing concentrations of LMWuPA:PAI-1 complex (3.12, 6.25, 12.5, 25, 50 nM) were injected over the LRP1-coupled chip. The dissociation of each concentration was measured from the SPR data, with the initial value at t=0 normalized to 100%. B. Increasing concentrations of LMWuPA:PAI-1 (0.78, 1.56, 3.12, 6.25, 12.5, 25 and 50 nM) were injected over the LRP1-coupled chip. Fits of the experimental data (black lines) to a model including Schemes I and II are shown (blue lines). The data are representative of three independent experiments.

FIG. 8. Kinetic analysis of LMWuPA:PAI-1 complexes binding to cluster IV of LRP1. A) Increasing concentrations of uPA:PAI-1 complex (0.6, 1.2, 2.5, 5, 10, 20 and 40 nM) were injected over the LRP1-coupled chip. The dissociation of each concentration was measured from the SPR data, with the initial value at t=0 normalized to 100%. B) Increasing concentrations of LMWuPA:PAI-1 (0.6, 1.2, 2.5, 5, 10, 20 and 40 nM) were injected over the LRP1-coupled chip. Fits of the experimental data (black lines) to a Schemes I and II model (blue lines). The data are representative of three independent experiments.

FIG. 9. LRP1-mediated cellular uptake of LMWuPA:PAI-1 is reduced when complex is formed with PAI-1 containing mutations in lysine residues. 5 nM of 125I-labeled LMWuPA:PAI-1 complexes formed with I91L PAI-1 or the indicated mutant PAI-1 molecules were incubated with WI-38 human fibroblasts for 6 h at 37° C. in the absence or presence of excess RAP. Following incubation, the amount of internalized complex was quantified. The experiments were performed in triplicate.

FIG. 10. Wild type PAI-1 nucleic acid sequence (SEQ ID NO: 1); wild type PAI-1 amino acid sequence (SEQ ID NO: 2); and mature wild type PAI-1 amino acid sequence (SEQ ID NO: 3) is provided.

FIG. 11. Mature variant PAI-1 nucleic acid sequence (SEQ ID NO: 4) encoding a polypeptide having the following mutations within wild-type human mature PAI-1 amino acid sequence (SEQ ID NO: 3): K69A, K80A, K88A, I91L, R101A, K122A, Q123K, K176A, K207A, K263A, V343A, and R346V; and mature variant PAI-1 amino acid sequence (SEQ ID NO: 5) having the following mutations within wild-type human mature PAI-1 amino acid sequence (SEQ ID NO: 3): K69A, K80A, K88A, I91L, R101A, K122A, Q123K, K176A, K207A, K263A, V343A, and R346V are provided.

FIG. 12. Mature variant PAI-1/Fc nucleic acid sequence (SEQ ID NO: 6) encoding a polypeptide having the following mutations within wild-type human mature PAI-1 amino acid sequence (SEQ ID NO: 3): I91L, R101A, Q123K, V343A, R346V; and mature variant PAI-1/Fc amino acid sequence (SEQ ID NO: 7) having the following mutations within wild-type human mature PAI-1 amino acid sequence (SEQ ID NO: 3): I91L, R101A, Q123K, V343A, R346V are provided.

FIG. 13 shows that MDI-1001 targets inflammatory nets better than Aralast.

FIG. 14 shows an in vitro comparison between Aralast, Avelestat, MDI-1002, MDI-1003, and MDI-1004.

FIG. 15 shows that MDI-1003 targets NETs in CF sputum.

FIG. 16 shows elastase activity as a function of inhibitor concentration.

FIG. 17 shows that MDI-1002 protects against acute lung injury.

FIG. 18 shows that MDI-1002 protects against lung fibrosis.

FIG. 19 shows that MDI-1002 does not improve recovery after bleomycin.

FIG. 20 shows that inhaled MDI-1003 protects against acute lung injury.

FIG. 21 shows that MDI-1003 protects against lung fibrosis better than MDI-1001.

FIG. 22 shows that MDI-1003 improves recovery after bleomycin.

FIG. 23 shows an Fc-fusion construct for MDI-1002 and MDI-1004.

FIG. 24 shows Fc-fusion expression of MDI-1002 and MDI-1004.

FIG. 25 shows that Fc-fusion improves PK.

FIG. 26 shows that mutation of the LRP1 binding residues does affect the inhibition of neutrophil elastase in the presence of DNA NETs, thereby demonstrating reduced interaction with the clearance receptor, LRP1, and retention of activity against elastase in NETs.

### DEFINITIONS

As used herein, the term “Fc domain” refers to a dimer of two Fc domain monomers. An Fc domain has at least 80% sequence identity (e.g., at least 85%, 90%, 95%, 97%, or 100% sequence identity) to a human Fc domain that includes at least a C_{H}2 domain and a C_{H}3 domain. An Fc domain monomer includes second and third antibody constant domains (C_{H}2 and C_{H}3). In some embodiments, the Fc domain monomer also includes a hinge domain. An Fc domain does not include any portion of an immunoglobulin that is capable of acting as an antigen-recognition region, e.g., a variable domain or a complementarity determining region (CDR). In the wild-type Fc domain, the two Fc domain monomers dimerize by the interaction between the two C_{H}3 antibody constant domains, as well as one or more disulfide bonds that form between the hinge domains of the two dimerizing Fc domain monomers. In some embodiments, an Fc domain may be mutated to lack effector functions, typical of a “dead Fc domain.” In certain embodiments, each of the Fc domain monomers in an Fc domain includes amino acid substitutions in the C_{H}2 antibody constant domain to reduce the interaction or binding between the Fc domain and an Fcγ receptor. In some embodiments, the Fc domain contains one or more amino acid substitutions that reduce or inhibit Fc domain dimerization. An Fc domain can be any immunoglobulin antibody isotype, including IgG, IgE, IgM, IgA, or IgD. Additionally, an Fc domain can be an IgG subtype (e.g., IgG1, IgG2a, IgG2b, IgG3, or IgG4). The Fc domain can also be a non-naturally occurring Fc domain, e.g., a recombinant Fc domain.

As used herein, the term “fused” or “attached” is used to describe the combination or attachment of two or more elements, components, or protein domains, e.g., peptides or polypeptides, by means including chemical conjugation, recombinant means, and chemical bonds, e.g., amide bonds. For example, two single peptides in tandem series can be fused to form one contiguous protein structure, e.g., a polypeptide, through chemical conjugation, a chemical bond, a peptide linker, or any other means of covalent linkage.

As used herein, the term “polypeptide” describes a single polymer in which the monomers are amino acid residues which are covalently conjugated together through amide bonds. A polypeptide is intended to encompass any amino acid sequence, either naturally occurring, recombinant, or synthetically produced.

As used herein, the term “homodimer” refers to a molecular construct formed by two identical macromolecules, such as proteins or nucleic acids. The two identical monomers may form a homodimer by covalent bonds or non-covalent bonds. For example, an Fc domain may be a homodimer of two Fc domain monomers if the two Fc domain monomers contain the same sequence. In another example, a polypeptide described herein including a PAI-1 variant fused to an Fc domain monomer may form a homodimer through the interaction of two Fc domain monomers, which form an Fc domain in the homodimer.

As used herein, the term “heterodimer” refers to a molecular construct formed by two different macromolecules, such as proteins or nucleic acids. The two monomers may form a heterodimer by covalent bonds or non-covalent bonds. For example, a polypeptide described herein including a PAI-1 variant fused to an Fc domain monomer may form a heterodimer through the interaction of two Fc domain monomers, each fused to a different PAI-1 variant, which form an Fc domain in the heterodimer.

As used herein, the term “host cell” refers to a vehicle that includes the necessary cellular components, e.g., organelles, needed to express proteins from their corresponding nucleic acids. The nucleic acids are typically included in nucleic acid vectors that can be introduced into the host cell by conventional techniques known in the art (transformation, transfection, electroporation, calcium phosphate precipitation, direct microinjection, etc.). A host cell may be a prokaryotic cell, e.g., a bacterial cell, or a eukaryotic cell, e.g., a mammalian cell (e.g., a CHO cell or a HEK293 cell).

As used herein, the term “therapeutically effective amount” refers an amount of a polypeptide, nucleic acid, or vector of the invention or a pharmaceutical composition containing a polypeptide, nucleic acid, or vector of the invention effective in achieving the desired therapeutic effect in treating a patient having a disease, such as any condition characterized with aberrant NE activity and/or deficient A1AT activity (e.g., IPF, COPD, cystic fibrosis, emphysema, ARDS, ischemia reperfusion, chronic pancreatitis, RA, DIC, UC, Chron's disease, dermatological diseases). The term “therapeutically effective amount” also refers an amount of a polypeptide, nucleic acid, or vector of the invention or a pharmaceutical composition containing a polypeptide, nucleic acid, or vector of the invention effective in achieving the desired therapeutic effect in treating a patient having such a condition. In particular, the therapeutically effective amount of the polypeptide, nucleic acid, or vector avoids adverse side effects.

As used herein, the term “pharmaceutical composition” refers to a medicinal or pharmaceutical formulation that includes an active ingredient as well as excipients and diluents to enable the active ingredient suitable for the method of administration. The pharmaceutical composition of the present invention includes pharmaceutically acceptable components that are compatible with the polypeptide, nucleic acid, or vector. The pharmaceutical composition may be in tablet or capsule form for oral administration or in aqueous form for intravenous or subcutaneous administration.

As used herein, the term “pharmaceutically acceptable carrier or excipient” refers to an excipient or diluent in a pharmaceutical composition. The pharmaceutically acceptable carrier must be compatible with the other ingredients of the formulation and not deleterious to the recipient. In the present invention, the pharmaceutically acceptable carrier or excipient must provide adequate pharmaceutical stability to the polypeptide including a PAI-1 variant, the nucleic acid molecule(s) encoding the polypeptide, or a vector containing such nucleic acid molecule(s). The nature of the carrier or excipient differs with the mode of administration. For example, for intravenous administration, an aqueous solution carrier is generally used; for oral administration, a solid carrier is preferred.

As used herein, the term “treating and/or preventing” refers to the treatment and/or prevention of a disease, e.g., any condition characterized with aberrant NE activity and/or deficient A1AT activity (e.g., IPF, COPD, cystic fibrosis, emphysema), using methods and compositions of the invention. Generally, treating such a disease occurs after a subject has developed the disease and/or is already diagnosed with the disease. Preventing such a disease refers to steps or procedures taken when a subject is at risk of developing the disease. The subject may show signs or mild symptoms that are judged by a physician to be indications or risk factors for developing the disease or have a family history or genetic predisposition of developing the disease, but has not yet developed the disease.

As used herein, the term “subject” refers to a mammal, e.g., preferably a human. Mammals include, but are not limited to, humans and domestic and farm animals, such as monkeys, mice, dogs, cats, horses, and cows, etc.

### DETAILED DESCRIPTION OF THE INVENTION

IPF is characterized by interstitial scar tissue formation that can dramatically restrict lung function. There are approximately 130,000 IPF patients in the US with an estimated 30,000 to 40,000 new cases diagnosed annually (see, Ley, B., and Collard, H. R. 2013. Epidemiology of idiopathic pulmonary fibrosis. Clin. Epidemiol. 5:483-492; Lynch, J. P., III, et al., 2016. Idiopathic Pulmonary Fibrosis: Epidemiology, Clinical Features, Prognosis, and Management. Semin. Respir. Crit Care Med. 37:331-357). Life expectancy following a diagnosis of IPF is generally three to five years (see, Lederer, D. J., and Martinez, F. J. NEJM 2018; 378:1811-1823). There is no effective therapeutic treatment except lung transplant. The lack of therapies that halt the progression of IPF presents a significant challenge and is a major unmet medical need. The two currently approved drugs, pirfenidone and nintedanib, have been shown to slow disease progression but they do not stop progression and cannot restore lost lung function. As such, a novel therapeutic for IPF treatment is necessary for disease management.

The present invention addresses this need through providing compositions comprising mutant PA1-I polypeptides capable of inhibiting NE, and in particular, inhibiting NE bound in NETs.

The present invention addresses this need through providing compositions comprising mutant PA1-I polypeptides capable of inhibiting NE, and in particular, inhibiting NE bound in NETs. Provided herein are PAI-1 variants capable of inhibiting NE activity while having a diminished ability to bind with vitronectin and/or LRP1. Indeed, experiments conducted during the course of developing embodiments for the present invention demonstrated such PAI-1 variants to have improved efficacy for treating conditions associated with NE activity (e.g., IPF) through inhibiting its ability to bind with vitronectin through modifying the PAI-1 amino acid residues responsible for such vitronectin binding (e.g., R101A and Q123K).

Experiments conducted during the course of developing embodiments for the present invention utilized a serpin mapping technology and identified mutant forms of PA1-I (e.g., having one or more of the following mutations within wild-type human mature PAI-1 amino acid sequence (SEQ ID NO: 3): K69A, K80A, K88A, I91L, R101A, K122A, Q123K, K176A, K207A, K263A, V343A, and R346V) as an inhibitor of NE capable of efficient inhibition of NE within the NETs environment. Such mutant forms of PA1-I were shown to irreversibly inhibit NE bound to DNA, a major component of NETs, where the FDA approved human plasma derived A1AT trademarked Aralast is ineffective. In certain embodiments, such mutant forms of PAI-1 are further associated with the human IgG-Fc.

As part of the innate immune response, neutrophils are the most abundant leukocytes in the peripheral blood, and are at the forefront of defense against infection. Neutrophils efficiently clear microbial infections by phagocytosis and by oxygen-dependent and oxygen-independent mechanisms. Recently, a new neutrophil anti-microbial mechanism was described, the release of NETs composed of DNA, histones and antimicrobial peptides. Such mutant forms of PAI-1 represent a first therapeutic to specifically target NE which is responsible for a significant amount of lung function loss in IPF (see, Obayashi, Y., et al., 1997Chest 112:1338-1343; Schaaf, B., et al., 2000 Respiration 67:52-59; Takemasa, A., et al., 2012 Eur. Respir. J 40:1475-1482; Kristensen, J. H., et al., 2015 BMC. Pulm. Med. 15:53) and other destructive lung diseases (see, Gregory, A. D., et al., 2015 J Leukoc. Biol. 98:143-152) (e.g., cystic fibrosis and chronic obstructive pulmonary disease (COPD)). The prospect of a more effective treatment and potential disease reversal would be highly attractive to patients suffering from this orphan disease.

Clinical indications for such variant forms of PAT-1 include idiopathic pulmonary fibrosis (as NE-NETs are involved in the etiology of fibrosis, including differentiation of lung fibroblasts), COPD (see, Grabcanovic-Musija, F., et al., 2015 Respir. Res. 16:59), cystic fibrosis which represent pulmonary diseases in which NE-NETs are elevated and where current treatment options are limited once these diseases are established, emphysema, ARDS, ischemia reperfusion, chronic pancreatitis, RA, DIC, UC, Chron's disease, and dermatological diseases.

Accordingly, the present invention features polypeptides that include PAI-1 variants capable of inhibiting NE activity while having a diminished ability to bind with vitronectin and LRP1 through modifying its amino acid residues responsible for vitronectin binding (e.g., R101A and Q123K) and/or LRP1 binding (e.g., K207, K88 and K80) resulting in improved pharmacokinetics (PK) and improved efficacy for treating conditions associated with NE activity (e.g., IPF). In some embodiments, a polypeptide of the invention includes a PAI-1 variant fused to the N- or C-terminus of an Fc domain monomer or moiety (e.g., for purposes of improving PK). In some embodiments, a polypeptide of the invention includes a PAI-1 variant fused to the N- or C-terminus of an Fc domain monomer or moiety. In some embodiments, the Fc domain monomer or moiety increases stability or improves the pharmacokinetics of the polypeptide. A polypeptide including a PAI-1 variant fused to an Fc domain monomer may also form a dimer (e.g., homodimer or heterodimer) through the interaction between two Fc domain monomers. The PAI-1 variants described herein are capable of inhibiting NE activity and are capable of inhibiting NE activity wherein the NE is bound within NETs. The invention also includes methods of treating diseases and conditions involving aberrant NE activity and/or deficient A1AT activity in a subject by administering to the subject a polypeptide including a PAI-1 variant described herein.

Elastase is a serine proteinase released by activated neutrophils and macrophages and monocytes. During inflammatory responses, neutrophils are activated and release elastase leading to tissue destruction through proteolysis. In the lung, elastase degrades elastic tissues and leads to emphysema. Elastase is also a compounding factor in cystic fibrosis (CF) and in both adult and infant acute respiratory distress syndrome (ARDS). Elastase has also been implicated in TNF-mediated inflammation (see, Massague, J. et al., Annu. Rev. Biochem. 62:515-541 (1993) and HIV infection (Bristow, C. L. et al., International Immunol. 7:239-249 (1995)).

Elastase has a broader spectrum of reactivity than plasminogen activators each of which acts preferentially on a precursor substrate to activate it.

The natural defense to elastase is a protein called α₁ anti-trypsin (α₁AT) or α₁ proteinase inhibitor ((α₁PI). Patients who are deficient in α₁AT are prone to emphysema, especially smokers. Furthermore, smoking provokes inflammation. In such α₁AT deficiencies, the enzyme is present (CRM⁺) but is functionally impaired. In addition, even in individuals with normal enzyme, smoking directly inactivates α₁AT. Therefore, an improved inhibitor of elastase would be highly desirable for the prevention of emphysema in susceptible subjects or for reversal of the pathophysiological process leading to this and other related diseases.

The major PAIs belong to the serine proteinase inhibitor (serpin) gene superfamily which includes many proteinase inhibitors in blood as well as other proteins with unrelated or unknown function (see, Gettins, P. G. W., and Olson, S. T. (2016) Inhibitory serpins. New insights into their folding, polymerization, regulation and clearance. Biochem. J. 473, 2273-2293). The serpins share a common tertiary structure and have evolved from a common ancestor. Serpins regulate many processes including coagulation, fibrinolysis, complement activation, ovulation, angiogenesis, inflammation, neoplasia, viral pathogenesis and allergic reactivity.

Serpins act as suicide inhibitors, reacting only once with their target proteinase to form a sodium dodecyl sulfate (SDS)-stable complex. These complexes can dissociate to yield free active enzyme together with a cleaved inhibitor similar to that seen in the α₁AT crystal structure (see, Gettins, P. G. W., and Olson, S. T. (2016) Inhibitory serpins. New insights into their folding, polymerization, regulation and clearance. Biochem. J. 473, 2273-2293).

PAI-1 is considered one of the principal regulators of the PA system. It is a single chain glycoprotein with a molecular weight of 50 kDa (see, Van Mourik J A et al., J Biol Chem (1984) 259:14914-14921) and is the most efficient inhibitor known of the single- and two-chain forms of tPA and of uPA (see, Lawrence D et al., Eur J Biochem (1989) 186:523-533). PAI-1 also inhibits plasmin and trypsin (see, Hekman C M et al., Biochemistry (1988) 27:2911-2918) and also inhibits thrombin and activated protein C, though with much lower efficiency.

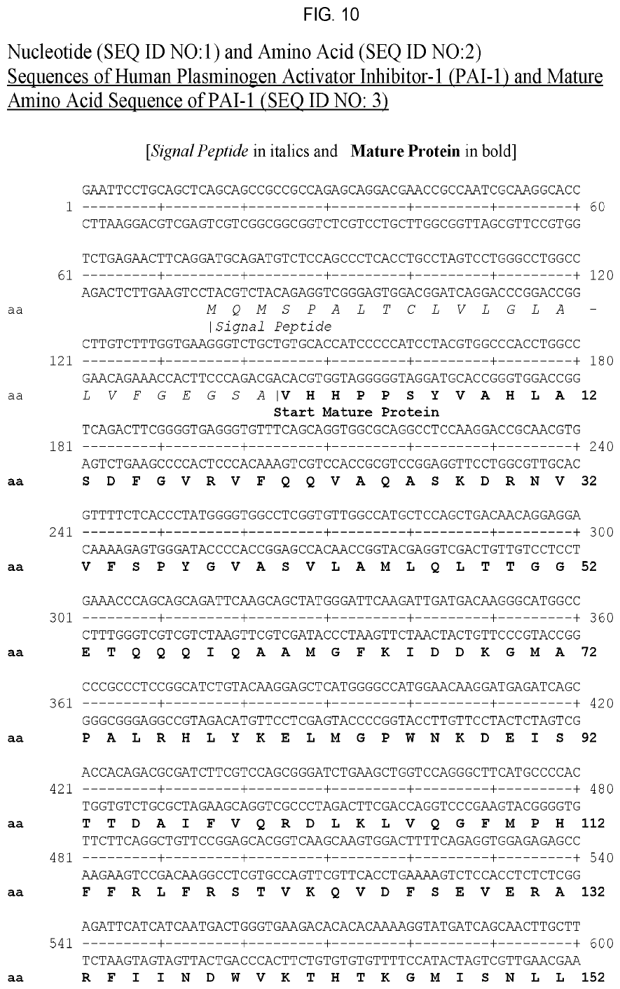

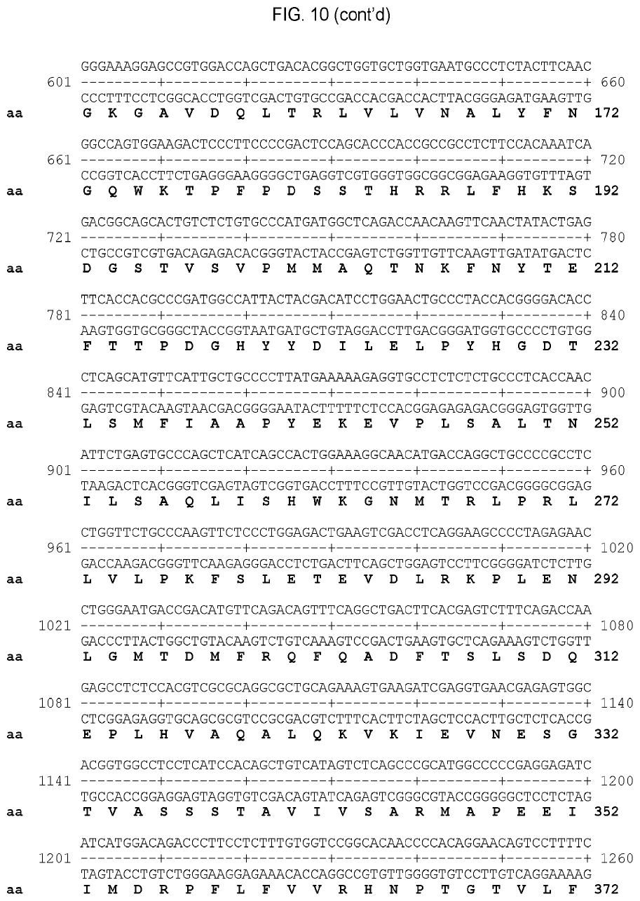

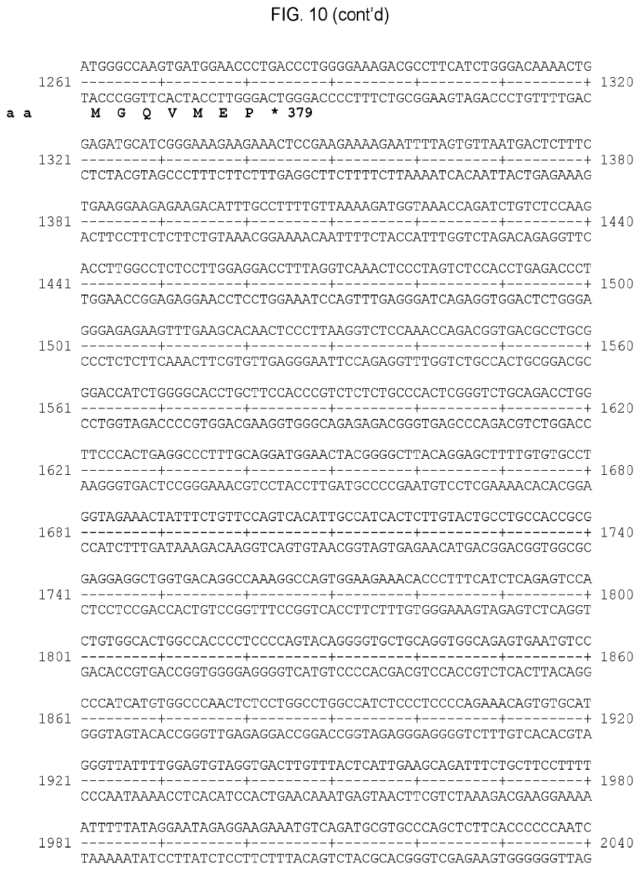

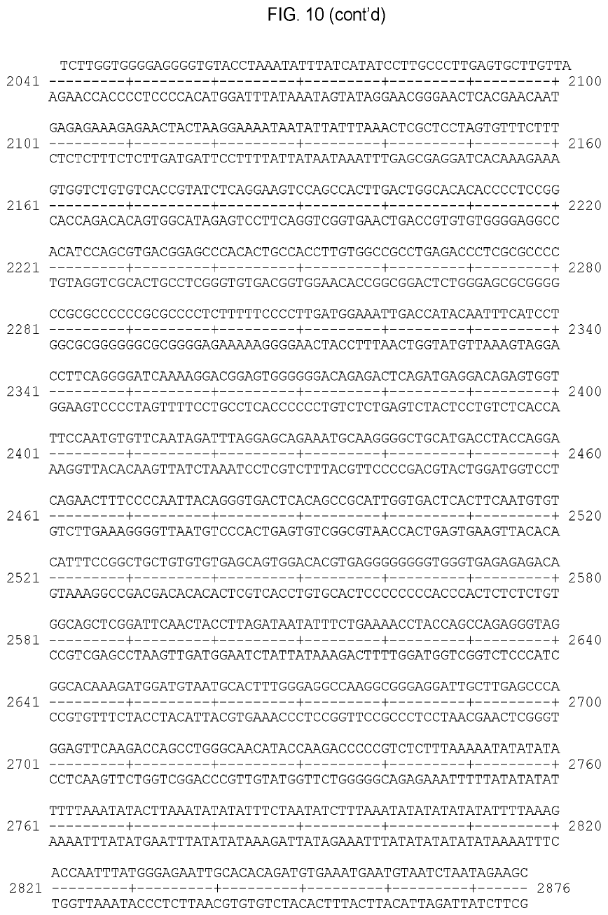

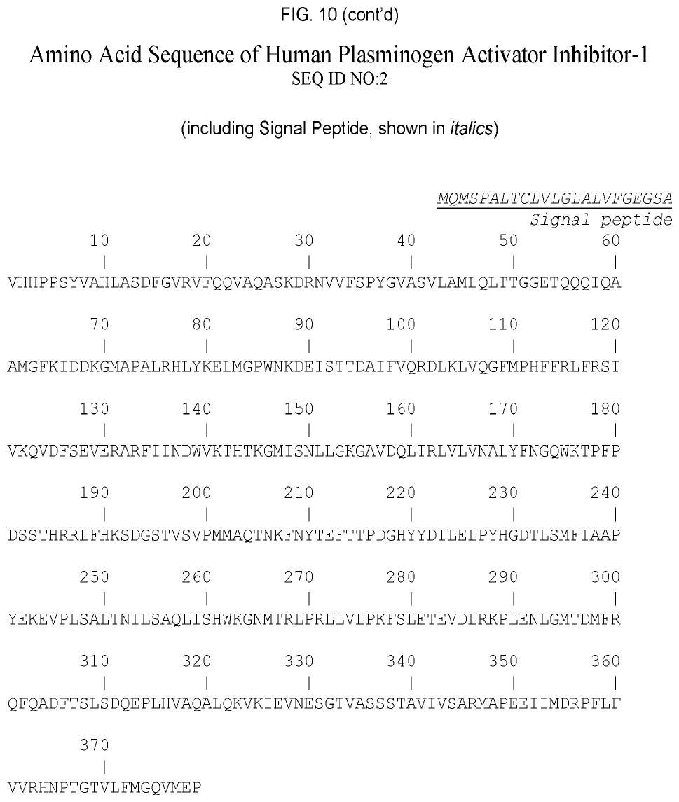

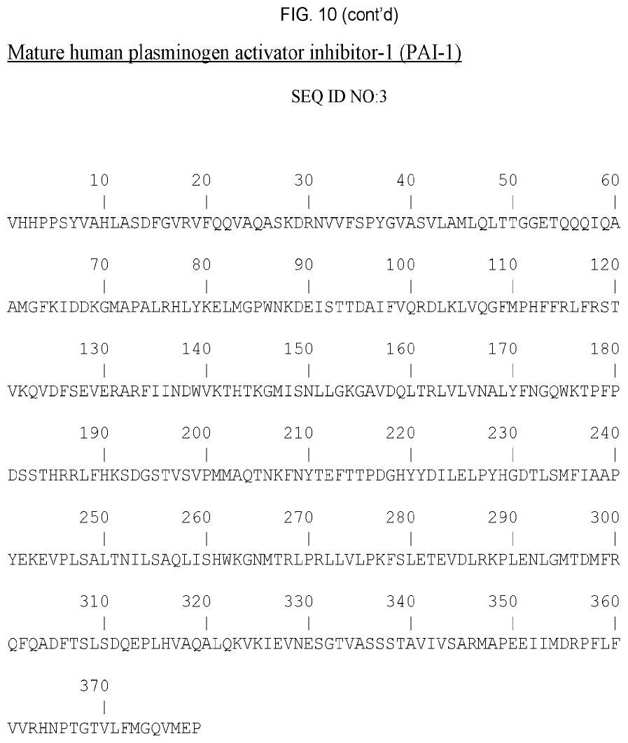

PAI-1 cDNA encodes a protein of 402 amino acids that includes a typical secretion signal sequence (see, Ny et al., supra; Ginsburg et al., 1986, supra). Mature human PAI-1 isolated from cell culture is composed of two variants of 381 and 379 amino acids in approximately equal proportions. FIG. 10 provides a human wild type PAI-1 nucleic acid sequence (SEQ ID NO: 1); human wild type PAI-1 amino acid sequence (SEQ ID NO: 2); and human mature wild type PAI-1 amino acid sequence (SEQ ID NO: 3).

PAI-1 is a glycoprotein with three potential N-linked glycosylation sites containing between 15 and 20% carbohydrate (Van Mourik J A et al., supra). Mature PAI-1 contains no cysteine residues, facilitating efficient expression and isolation of recombinant PAI-1 from E. coli. PAI-1 produced in E. coli, although nonglycosylated, is functionally very similar to native PAI-1. Recombinant PAI-1 can be isolated from E. coli in an inherently active form (see below), which contrasts with PAI-1 purified from mammalian cell culture (Lawrence et al., 1989, supra; Hekman et al., 1988, supra).

PAI-1 exists in an active form as it is produced by cells and secreted into the culture medium and an inactive or latent form that accumulates in the culture medium over time (see, Hekman C M et al, J Biol Chem (1985) 260:11581-11587, Levin E G et al, Blood (1987) 70:1090-1098). The active form spontaneously converts to the latent form with a half-life of about 1 h at 37° C. (see, Lawrence et al., supra, Hekman et al., supra; Levin E G et al, 1987, supra).

The latent form can be converted into the active form by treatment with denaturants, negatively charged phospholipids or Vn (see, Lambers et al, supra, Hekman et al, supra; Wun T-C et al, J Biol Chem (1989) 264:7862-7868). Latent PAI-1 infused into rabbits became reactivated in vivo by an unknown mechanism. The reversible interconversion between the active and latent structures, presumably due to a conformational change, is a unique feature of PAI-1 as compared to other serpins. The latent form appears to be more energetically favored.

The three-dimensional structure of the latent form of PAI-1 has been solved. In this structure the entire N-terminal side of the reactive center loop is inserted as the central strand into β sheet A (see, Mottonen et al., supra) which explains the increased stability (see, Lawrence, D. A. et al., Biochemistry 33:3643-3648 (1994)) as well as the lack of inhibitory activity.

The activity of the two plasminogen activators, urokinase-type plasminogen activator (uPA) and tissue-type plasminogen activator are regulated by plasminogen activator inhibitor 1 (PAI-1), a serine proteinase inhibitor (serpin) that regulates fibrinolysis and wound healing, and is associated with thrombotic and fibrotic disease. Serpins function to inhibit serine proteases by a unique mechanism following cleavage of the serpin's reactive center loop which induces a conformational change in the serpin resulting in protease inhibition (for review see (see, Gettins, P. G. W., and Olson, S. T. (2016) Biochem. J. 473, 2273-2293). Once a serpin complexes with a protease, the complex is rapidly removed from the circulation in the liver by binding to the LDL receptor related protein 1 (LRP1) (see, Kounnas, M. Z., Church, F. C., Argraves, W. S., and Strickland, D. K. (1996) J. Biol. Chem. 271, 6523-6529).

LRP1 was originally identified as the hepatic receptor responsible for the removal of alpha₂-macroglobulin protease complexes (see, Ashcom, J. D., et al., (1990) J. Cell Biol. 110, 1041-1048; Moestrup, S. K., and Gliemann, J. (1989) J. Biol. Chem. 264, 15574-15577) and as a receptor for chylomicron remnant lipoprotein particles (see, Rohlmann, A., et al., (1998) J. Clin. Invest. 101, 689-695). In addition to its endocytic role, LRP1 also regulates various signaling pathways (see, Gonias, S. L. (2018) Arter. Thromb Vasc Biol. 38, 2548-2549; Strickland, D. K., et al., (2014) Thromb. Vasc. Biol. 34, 487-498). The ectodomain of this large receptor is made up of modules that consist of clusters of LDLa repeats, EGF-like repeats and β-propeller domains. The efficient delivery of newly synthesized LRP1 to the cell surface requires the participation of an endoplasmic reticulum resident chaperone, termed the receptor associated protein (RAP) (see, Strickland, D. K., et al., (1991) J. Biol. Chem. 266, 13364-13369; Willnow, T. E., et al., (1995) Proc. Natl. Acad. Sci. U.S.A 92, 4537-41; Bu, G., et al., (1995) EMBO J. 14, 2269-80

The fact that LRP1 recognizes numerous structurally unrelated ligands with relatively high affinity has raised questions regarding the nature of ligand/receptor interaction. Insight into how this might occur resulted from recognition that K256 and K270 are essential for the third domain of RAP to bind LRP1 (see, Migliorini, M. M., et al., (2003) J. Biol. Chem. 278, 17986-17992) and from a crystal structure of the third domain of RAP in complex with two LDLa repeats from the LDL receptor (see, Fisher, C., et al., (2006) Mol. Cell. 22, 277-283). These studies revealed that the ε-amino groups of K256 and K270 on RAP form salt-bridges with carboxylates of aspartate residues within the LDLa repeats which form an acidic pocket on the receptor. To date several ligands including alpha2-macroglobulin (α₂M) (see, Arandjelovic, S., Hall, B. D., and Gonias, S. L. (2005) Arch. Biochem. Biophys. 438, 29-35) and blood coagulation factor VIII (see, van den Biggelaar, et al., (2015) J. Biol. Chem. 290, 16463-76; Young, P. A., et al., (2016) J. Biol. Chem. 291, 26035-26044) interact with LRP1 via interactions involving critical lysine residues.

While lysine residues appear to contribute to the interaction of PAI-1 with LRP1 (see, Horn, I., et al., (1998) Thromb. Haemost. 1, 20-22; Rodenburg, K. W., et al., (1998) Biochem. J. 329, 55-63; Gettins, P. G. W., and Dolmer, K. (2016) J. Biol. Chem. 291, 800-812), studies investigating the interaction of PAI-1 with LRP1 have resulted in conflicting data. First, questions exist regarding the relative affinity of PAI-1 vs protease: PAI-1 complexes to LRP1. Most studies have demonstrated that only PAI-1 in complex with a protease binds to LRP1 with high affinity (see, Horn, I., et al., (1998) Thromb. Haemost. 1, 20-22; Stefansson, S. (1998) J. Biol. Chem. 273, 6358-6366; Nykjaer, A., et al., (1992) J. Biol. Chem. 267, 14543-14546; Horn, I. R., et al., (1997) J. Biol. Chem. 272, 13608-13613). In contrast, other studies (see, Gettins, P. G. W., and Dolmer, K. (2016) J. Biol. Chem. 291, 800-812; Jensen, J. K., et al., (2009) J. Biol. Chem. 284, 17989-17997) have reported that PAI-1 alone binds with high affinity to fragments derived from LRP1. Secondly, based on the observation that protease:PAI-1 complexes binds with higher affinity to LRP1 than PAI-1 alone, some have proposed that formation of a protease complex with PAI-1 exposes a cryptic epitope on PAI-1 that is recognized by LRP1 (see, Horn, I., et al., (1998) Thromb. Haemost. 1, 20-22; Stefansson, S. (1998) J. Biol. Chem. 273, 6358-6366). In contrast, others have argued that the protease itself might interact with LRP1 and contribute to the high affinity interaction (see, Skeldal, S., et al., (2006) FEBS J. 273, 5143-5159). Finally, while a number of studies have reported changes in the affinity of PAI-1 for LRP1 when various basic residues are mutated to alanine (see, Horn, I., et al., (1998) Thromb. Haemost. 1, 20-22; Stefansson, S. (1998) J. Biol. Chem. 273, 6358-6366; Skeldal, S., et al., (2006) FEBS J. 273, 5143-5159), there seems to be little consensus on which lysine residues constitute the binding site.

Experiments conducted during the course of developing embodiments of the present invention clarified the relative binding affinities of PAI-1 and protease:PAI-1 complexes with LRP1. In addition, experiments were conducted to determine if the binding of a protease:PAI-1 complex to LRP1 is mainly attributed to determinants on PAI-1. In addition, experiments were conducted to determine the specific amino acid residues in free PAI-1 and PAI-1 in complex with a target protease (urokinase-type plasminogen activator (uPA)) that participate in their binding to LRP1. Indeed, mutational analysis revealed overlap between LRP1 binding and the binding site for a small molecule inhibitor of PAI-1, CDE-096, with an important role for K207 in the interaction of PAI-1 with LRP1 and K207, K88 and K80 for the interaction of uPA:PAI-1 complexes with LRP1.

Experiments conducted during the course of developing embodiments for the present invention utilized a serpin mapping technology and identified mutant forms of PA1-I (e.g., having one or more of the following mutations within wild-type human mature PAI-1 amino acid sequence (SEQ ID NO: 3): K69A, K80A, K88A, I91L, R101A, K122A, Q123K, K176A, K207A, K263A, V343A, and R346V) as an inhibitor of NE capable of efficient inhibition of NE within the NETs environment. Such mutant forms of PA1-I were shown to irreversibly inhibit NE bound to DNA, a major component of NETs, where the FDA approved human plasma derived A1AT trademarked Aralast is ineffective. In certain embodiments, such mutant forms of PAI-1 are further associated with the human IgG-Fc.

Accordingly, the present invention features polypeptides that include an PAI-1 variant. In some embodiments, a polypeptide of the invention includes a PAI-1 variant fused to the N- or C-terminus of an Fc domain monomer or moiety. In some embodiments, a polypeptide of the invention includes a PAI-1 variant fused to the N- or C-terminus of an Fc domain monomer or moiety. In some embodiments, the Fc domain monomer or moiety increases stability or improves the pharmacokinetics of the polypeptide.

Such moieties may be fused or attached by amino acid or other covalent bonds and may increase stability of the polypeptide. A polypeptide including a PAI-1 variant fused to an Fc domain monomer may also form a dimer (e.g., a homodimer or heterodimer) through the interaction between two Fc domain monomers. In some embodiments, a polypeptide described herein attached with an Fc domain monomer is fused to the polypeptide by way of a linker. In some embodiments, the linker is an amino acid spacer.

The polypeptides of the invention may be used to inhibit NE activity and may be used to inhibit NE activity bound in NETs. In addition, the polypeptides of the invention may be used to treat a subject having a condition characterized with aberrant NE activity (e.g., IPF). In addition, the polypeptides of the invention may be used to prevent a subject from infliction of a condition characterized with aberrant NE activity (e.g., IPF). Further, the polypeptides of the invention may also be used to affect NE activity in a subject having a risk of developing or having a disease or condition involving aberrant NE activity.

In some embodiments, the invention features a polypeptide including PAI-1 variant, the variant having one or more of the following mutations within wild-type human mature PAI-1 amino acid sequence (SEQ ID NO: 3): K69A, K80A, K88A, I91L, R101A, K122A, Q123K, K176A, K207A, K263A, V343A, and R346V. In some embodiments, the PAI-1 variant includes mutations within wild-type human mature PAI-1 amino acid sequence (SEQ ID NO: 3): K69A, K80A, K88A, I91L, R101A, K122A, Q123K, K176A, K207A, K263A, V343A, and R346V as shown in SEQ ID NO: 5. In some embodiments, the PAI-1 variant includes the following mutations within wild-type human mature PAI-1 amino acid sequence (SEQ ID NO: 3): R101A and Q123K. In some embodiments, the PAI-1 variant includes the following mutations within wild-type human mature PAI-1 amino acid sequence (SEQ ID NO: 3): I91L, R101A, Q123K, V343A, R346V.

In some embodiments, the invention features a polypeptide including PAI-1 variant attached with an Fc domain monomer or moiety, the variant having one or more of the following mutations within wild-type human mature PAI-1 amino acid sequence (SEQ ID NO: 3): K69A, K80A, K88A, I91L, R101A, K122A, Q123K, K176A, K207A, K263A, V343A, and R346V. In some embodiments, the PAI-1 variant attached with an Fc domain monomer or moiety includes the following mutations within wild-type human mature PAI-1 amino acid sequence (SEQ ID NO: 3): K69A, K80A, K88A, I91L, R101A, K122A, Q123K, K176A, K207A, K263A, V343A, and R346V. In some embodiments, the PAI-1 variant attached with an Fc domain monomer or moiety includes the following mutations within wild-type human mature PAI-1 amino acid sequence (SEQ ID NO: 3): R101A and Q123K. In some embodiments, the PAI-1 variant attached with an Fc domain monomer or moiety includes the following mutations within wild-type human mature PAI-1 amino acid sequence (SEQ ID NO: 3): I91L, R101A, Q123K, V343A, R346V (SEQ ID NO: 7).

In some embodiments, the polypeptide described herein is capable of inhibiting NE, and in particular, inhibiting NE bound in NETs.

In some embodiments, the invention features a nucleic acid molecule encoding a polypeptide described herein (e.g., a polypeptide including a PAI-1 variant having one or more of the following mutations within wild-type human mature PAI-1 amino acid sequence (SEQ ID NO: 3): K69A, K80A, K88A, I91L, R101A, K122A, Q123K, K176A, K207A, K263A, V343A, and R346V. In another aspect, the invention also features a vector including the nucleic acid molecule described herein.

In another aspect, the invention features a host cell that expresses a polypeptide described herein, wherein the host cell includes a nucleic acid molecule or a vector described in the previous two aspects, wherein the nucleic acid molecule or vector is expressed in the host cell.

In another aspect, the invention features a method of preparing a polypeptide described herein, wherein the method includes: a) providing a host cell including a nucleic acid molecule or a vector described herein, and b) expressing the nucleic acid molecule or vector in the host cell under conditions that allow for the formation of the polypeptide.

In another aspect, the invention features a pharmaceutical composition including a polypeptide, nucleic acid molecule, or vector described herein and one or more pharmaceutically acceptable carriers or excipients. In some embodiments of the pharmaceutical composition, the polypeptide, nucleic acid molecule, or vector is in a therapeutically effective amount.

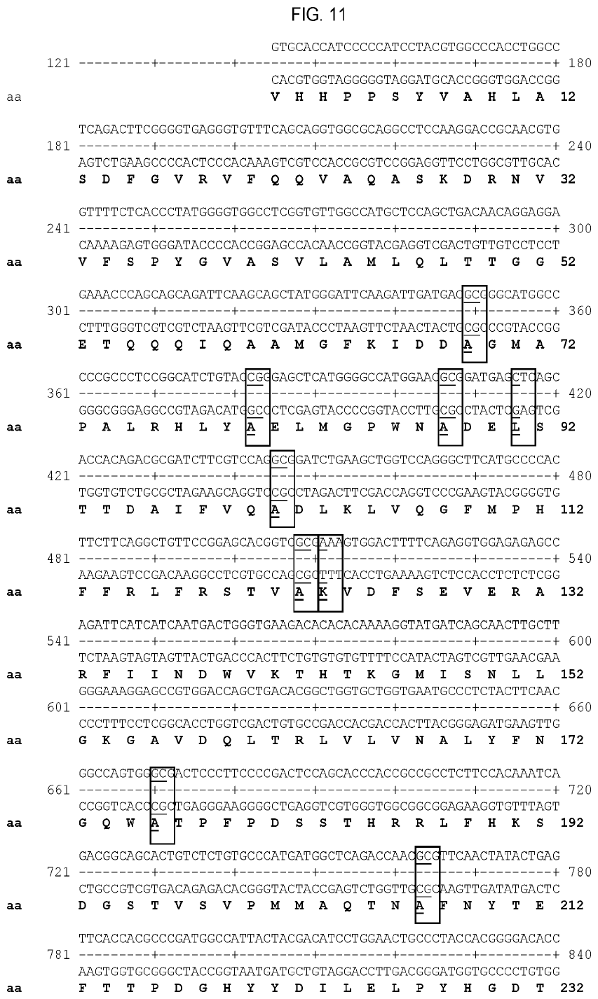

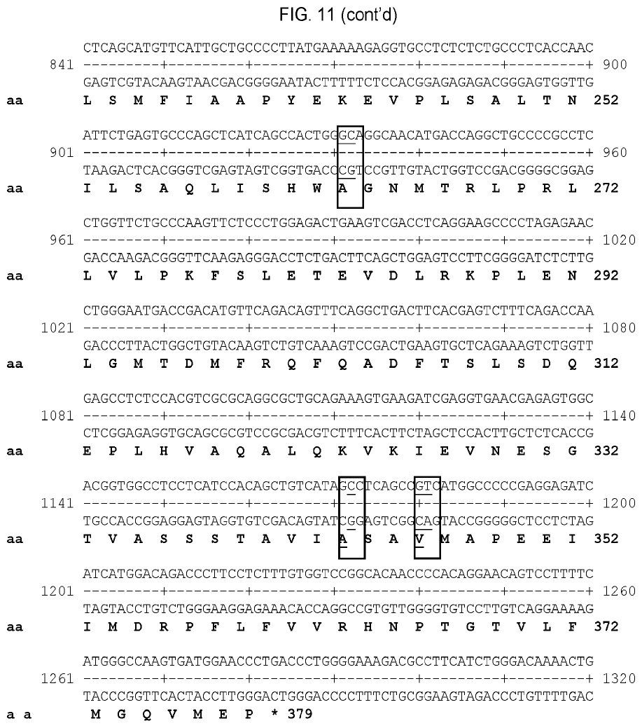

FIG. 11 provides a mature variant PAI-1 nucleic acid sequence (SEQ ID NO: 4) encoding a polypeptide having the following mutations within wild-type human mature PAI-1 amino acid sequence (SEQ ID NO: 3): K69A, K80A, K88A, I91L, R101A, K122A, Q123K, K176A, K207A, K263A, V343A, and R346V; and provides a mature variant PAI-1 amino acid sequence (SEQ ID NO: 5) having the following mutations within wild-type human mature PAI-1 amino acid sequence (SEQ ID NO: 3): K69A, K80A, K88A, I91L, R101A, K122A, Q123K, K176A, K207A, K263A, V343A, and R346V.

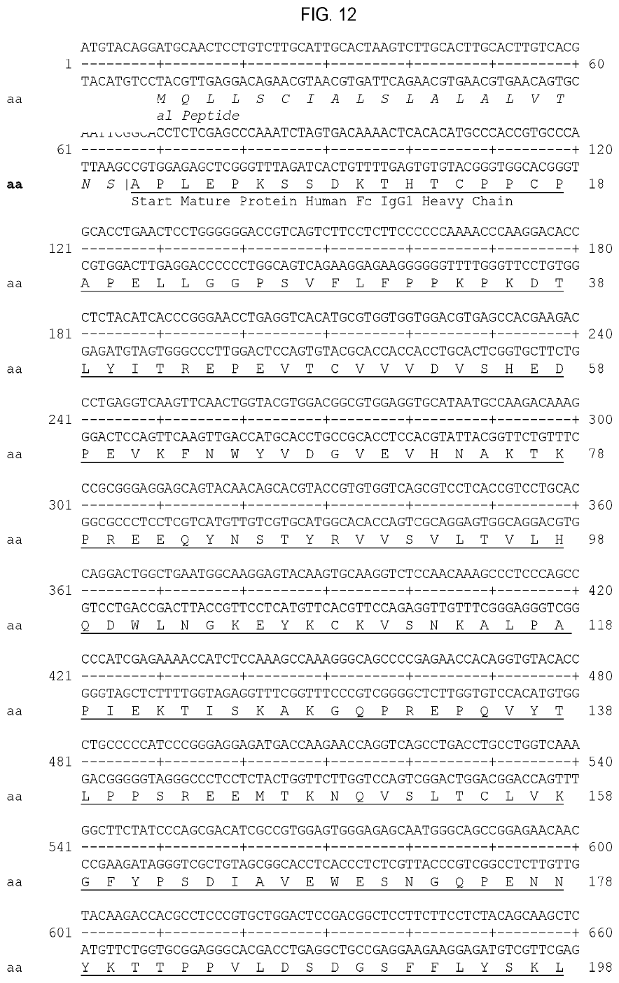

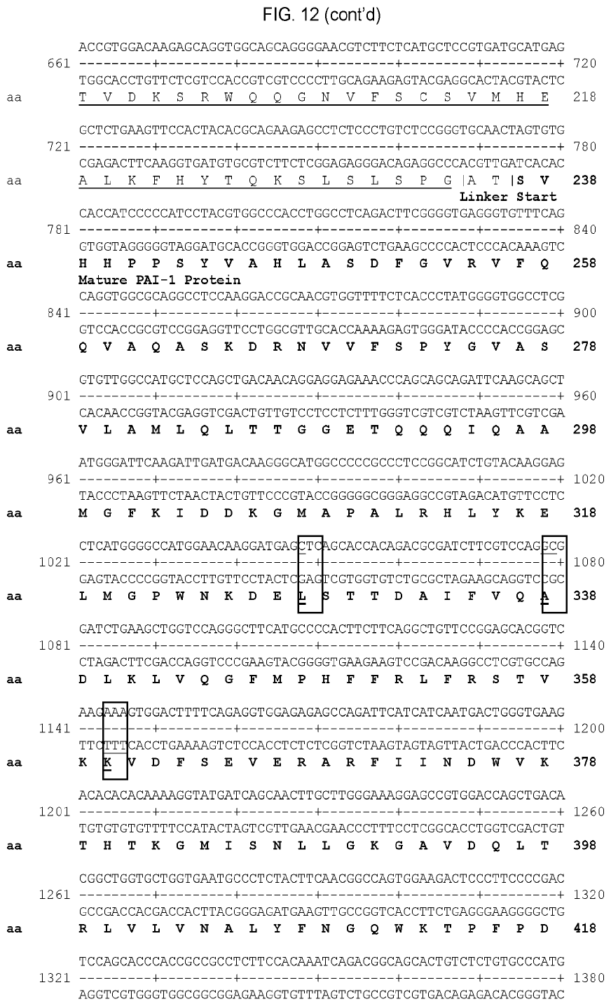

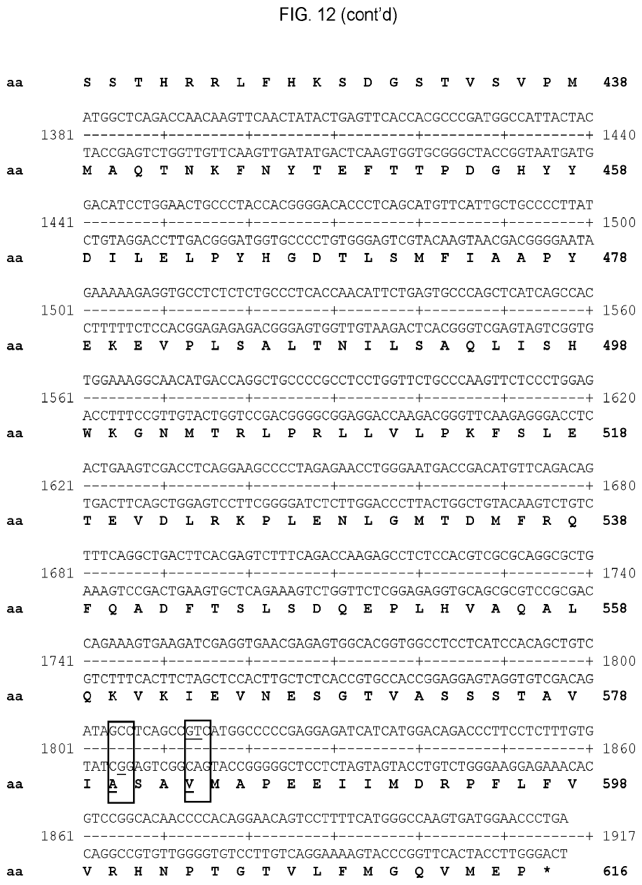

FIG. 12 provides a mature variant PAI-1/Fc nucleic acid sequence (SEQ ID NO: 6) encoding a polypeptide having the following mutations within wild-type human mature PAI-1 amino acid sequence (SEQ ID NO: 3): I91L, R101A, Q123K, V343A, R346V; and provides a mature variant PAI-1/Fc amino acid sequence (SEQ ID NO: 7) having the following mutations within wild-type human mature PAI-1 amino acid sequence (SEQ ID NO: 3): I91L, R101A, Q123K, V343A, R346V.

In some embodiments, a polypeptide described herein may include a PAI-1 variant fused to an Fc domain monomer of an immunoglobulin or a fragment of an Fc domain to increase the serum half-life of the polypeptide. A polypeptide including a PAI-1 variant fused to an Fc domain monomer may form a dimer (e.g., homodimer or heterodimer) through the interaction between two Fc domain monomers, which form an Fc domain in the dimer. As conventionally known in the art, an Fc domain is the protein structure that is found at the C-terminus of an immunoglobulin. An Fc domain includes two Fc domain monomers that are dimerized by the interaction between the C_{H}3 antibody constant domains. A wild-type Fc domain forms the minimum structure that binds to an Fc receptor, e.g., FcγRI, FcγRIIa, FcγRIIb, FcγRIIIa, FcγRIIIb, FcγRIV. In some embodiments, an Fc domain may be mutated to lack effector functions, typical of a “dead” Fc domain. For example, an Fc domain may include specific amino acid substitutions that are known to minimize the interaction between the Fc domain and an Fcγ receptor.

The polypeptides of the invention can be produced from a host cell. A host cell refers to a vehicle that includes the necessary cellular components, e.g., organelles, needed to express the polypeptides and fusion polypeptides described herein from their corresponding nucleic acids. The nucleic acids may be included in nucleic acid vectors that can be introduced into the host cell by conventional techniques known in the art (e.g., transformation, transfection, electroporation, calcium phosphate precipitation, direct microinjection, infection, or the like). The choice of nucleic acid vectors depends in part on the host cells to be used. Generally, preferred host cells are of either eukaryotic (e.g., mammalian) or prokaryotic (e.g., bacterial) origin.

A nucleic acid sequence encoding the amino acid sequence of a polypeptide of the invention may be prepared by a variety of methods known in the art. These methods include, but are not limited to, oligonucleotide-mediated (or site-directed) mutagenesis and PCR mutagenesis. A nucleic acid molecule encoding a polypeptide of the invention may be obtained using standard techniques, e.g., gene synthesis. Alternatively, a nucleic acid molecule encoding a wild-type PAI-1 may be mutated to include specific amino acid substitutions using standard techniques in the art, e.g., QuikChange™ mutagenesis. Nucleic acid molecules can be synthesized using a nucleotide synthesizer or PCR techniques.

A nucleic acid sequence encoding a polypeptide of the invention may be inserted into a vector capable of replicating and expressing the nucleic acid molecule in prokaryotic or eukaryotic host cells. Many vectors are available in the art and can be used for the purpose of the invention. Each vector may include various components that may be adjusted and optimized for compatibility with the particular host cell. For example, the vector components may include, but are not limited to, an origin of replication, a selection marker gene, a promoter, a ribosome binding site, a signal sequence, the nucleic acid sequence encoding protein of interest, and a transcription termination sequence.

In some embodiments, mammalian cells may be used as host cells for the invention. Examples of mammalian cell types include, but are not limited to, human embryonic kidney (HEK) (e.g., HEK293, HEK 293F), Chinese hamster ovary (CHO), HeLa, COS, PC3, Vero, MC3T3, NS0, Sp2/0, VERY, BHK, MDCK, W138, BT483, Hs578T, HTB2, BT20, T47D, NS0 (a murine myeloma cell line that does not endogenously produce any immunoglobulin chains), CRL7O3O, and HsS78Bst cells. In some embodiments, E. coli cells may also be used as host cells for the invention. Examples of E. coli strains include, but are not limited to, E. coli 294 (ATCC® 31,446), E. coli λ1776 (ATCC®31,537, E. coli BL21 (DE3) (ATCC® BAA-1025), and E. coli RV308 (ATCC®31,608). Different host cells have characteristic and specific mechanisms for the posttranslational processing and modification of protein products (e.g., glycosylation). Appropriate cell lines or host systems may be chosen to ensure the correct modification and processing of the polypeptide expressed. The above-described expression vectors may be introduced into appropriate host cells using conventional techniques in the art, e.g., transformation, transfection, electroporation, calcium phosphate precipitation, and direct microinjection. Once the vectors are introduced into host cells for protein production, host cells are cultured in conventional nutrient media modified as appropriate for inducing promoters, selecting transformants, or amplifying the genes encoding the desired sequences. Methods for expression of therapeutic proteins are known in the art, see, for example, Paulina Balbas, Argelia Lorence (eds.) Recombinant Gene Expression: Reviews and Protocols (Methods in Molecular Biology), Humana Press; 2nd ed. 2004 and Vladimir Voynov and Justin A. Caravella (eds.) Therapeutic Proteins: Methods and Protocols (Methods in Molecular Biology) Humana Press; 2nd ed. 2012.

Host cells used to produce the polypeptides of the invention may be grown in media known in the art and suitable for culturing of the selected host cells. Examples of suitable media for mammalian host cells include Minimal Essential Medium (MEM), Dulbecco's Modified Eagle's Medium (DMEM), Expi293™ Expression Medium, DMEM with supplemented fetal bovine serum (FBS), and RPMI-1640. Examples of suitable media for bacterial host cells include Luria broth (LB) plus necessary supplements, such as a selection agent, e.g., ampicillin. Host cells are cultured at suitable temperatures, such as from about 20° C. to about 39° C., e.g., from 25° C. to about 37° C., preferably 37° C., and CO₂ levels, such as 5 to 10%. The pH of the medium is generally from about 6.8 to 7.4, e.g., 7.0, depending mainly on the host organism. If an inducible promoter is used in the expression vector of the invention, protein expression is induced under conditions suitable for the activation of the promoter.

In some embodiments, depending on the expression vector and the host cells used, the expressed protein may be secreted from the host cells (e.g., mammalian host cells) into the cell culture media. Protein recovery may involve filtering the cell culture media to remove cell debris. The proteins may be further purified. A polypeptide of the invention may be purified by any method known in the art of protein purification, for example, by chromatography (e.g., ion exchange, affinity, and size-exclusion column chromatography), centrifugation, differential solubility, or by any other standard technique for the purification of proteins. For example, the protein can be isolated and purified by appropriately selecting and combining affinity columns such as Protein A column (e.g., POROS Protein A chromatography) with chromatography columns (e.g., POROS HS-50 cation exchange chromatography), filtration, ultra filtration, salting-out and dialysis procedures.

In other embodiments, host cells may be disrupted, e.g., by osmotic shock, sonication, or lysis, to recover the expressed protein. Once the cells are disrupted, cell debris may be removed by centrifugation or filtration. In some instances, a polypeptide can be conjugated to marker sequences, such as a peptide to facilitate purification. An example of a marker amino acid sequence is a hexa-histidine peptide (His-tag), which binds to nickel-functionalized agarose affinity column with micromolar affinity. Other peptide tags useful for purification include, but are not limited to, the hemagglutinin “HA” tag, which corresponds to an epitope derived from influenza hemagglutinin protein (see, Wilson et al., Cell 37:767, 1984).

Alternatively, the polypeptides of the invention can be produced by the cells of a subject (e.g., a human), e.g., in the context of gene therapy, by administrating a vector (such as a viral vector (e.g., a retroviral vector, adenoviral vector, poxviral vector (e.g., vaccinia viral vector, such as Modified Vaccinia Ankara (MVA)), adeno-associated viral vector, and alphaviral vector)) containing a nucleic acid molecule encoding the polypeptide of the invention. The vector, once inside a cell of the subject (e.g., by transformation, transfection, electroporation, calcium phosphate precipitation, direct microinjection, infection, etc.) will promote expression of the polypeptide, which is then secreted from the cell. If treatment of a disease or disorder is the desired outcome, no further action may be required. If collection of the protein is desired, blood may be collected from the subject and the protein purified from the blood by methods known in the art.

The invention features pharmaceutical compositions that include the polypeptides described herein (e.g., a polypeptide including a PAI-1 variant (e.g., a PAI-1 variant having one or more of the following mutations within wild-type PAI-1 (SEQ ID NO: 1): K69A, K80A, K88A, I91L, R101A, K122A, Q123K, K176A, K207A, K263A, V343A, and R346V. In some embodiments, a pharmaceutical composition of the invention includes a polypeptide including a PAI-1 variant with a C-terminal extension (e.g., 1, 2, 3, 4, 5, 6 or more additional amino acids) as the therapeutic protein. In some embodiments, a pharmaceutical composition of the invention includes a polypeptide including a PAI-1 variant fused to a moiety (e.g., Fc domain monomer, or a dimer thereof, a wild-type Fc domain, an Fc domain with amino acid substitutions (e.g., one or more substitutions that reduce dimerization)) as the therapeutic protein. In some embodiments, a pharmaceutical composition of the invention includes a polypeptide including a PAI-1 variant fused to a first moiety (e.g., Fc domain monomer, or a dimer thereof, a wild-type Fc domain, an Fc domain with amino acid substitutions (e.g., one or more substitutions that reduce dimerization)).

In some embodiments, a pharmaceutical composition of the invention including a polypeptide of the invention may be used in combination with other agents (e.g., therapeutic biologics and/or small molecules) or compositions in a therapy. In addition to a therapeutically effective amount of the polypeptide, the pharmaceutical composition may include one or more pharmaceutically acceptable carriers or excipients, which can be formulated by methods known to those skilled in the art. In some embodiments, a pharmaceutical composition of the invention includes a nucleic acid molecule (DNA or RNA, e.g., mRNA) encoding a polypeptide of the invention, or a vector containing such a nucleic acid molecule.

Acceptable carriers and excipients in the pharmaceutical compositions are nontoxic to recipients at the dosages and concentrations employed. Acceptable carriers and excipients may include buffers such as phosphate, citrate, HEPES, and TAE, antioxidants such as ascorbic acid and methionine, preservatives such as hexamethonium chloride, octadecyldimethylbenzyl ammonium chloride, resorcinol, and benzalkonium chloride, proteins such as human serum albumin, gelatin, dextran, and immunoglobulins, hydrophilic polymers such as polyvinylpyrrolidone, amino acids such as glycine, glutamine, histidine, and lysine, and carbohydrates such as glucose, mannose, sucrose, and sorbitol. Pharmaceutical compositions of the invention can be administered parenterally in the form of an injectable formulation. Pharmaceutical compositions for injection can be formulated using a sterile solution or any pharmaceutically acceptable liquid as a vehicle. Pharmaceutically acceptable vehicles include, but are not limited to, sterile water, physiological saline, and cell culture media (e.g., Dulbecco's Modified Eagle Medium (DMEM), α-Modified Eagles Medium (α-MEM), F-12 medium). Formulation methods are known in the art, see e.g., Banga (ed.) Therapeutic Peptides and Proteins: Formulation, Processing and Delivery Systems (3rd ed.) Taylor & Francis Group, CRC Press (2015).

The pharmaceutical compositions of the invention may be prepared in microcapsules, such as hydroxylmethylcellulose or gelatin-microcapsule and poly-(methylmethacrylate) microcapsule. The pharmaceutical compositions of the invention may also be prepared in other drug delivery systems such as liposomes, albumin microspheres, microemulsions, nano-particles, and nanocapsules. Such techniques are described in Remington: The Science and Practice of Pharmacy 22^{th} edition (2012). The pharmaceutical compositions to be used for in vivo administration must be sterile. This is readily accomplished by filtration through sterile filtration membranes.

The pharmaceutical compositions of the invention may also be prepared as a sustained-release formulation. Suitable examples of sustained-release preparations include semipermeable matrices of solid hydrophobic polymers containing the polypeptides of the invention. Examples of sustained release matrices include polyesters, hydrogels, polyactides, copolymers of L-glutamic acid and y ethyl-L-glutamate, non-degradable ethylene-vinyl acetate, degradable lactic acid-glycolic acid copolymers such as LUPRON DEPOT™, and poly-D-(−)-3-hydroxybutyric acid. Some sustained-release formulations enable release of molecules over a few months, e.g., one to six months, while other formulations release pharmaceutical compositions of the invention for shorter time periods, e.g., days to weeks.

The pharmaceutical composition may be formed in a unit dose form as needed. The amount of active component, e.g., a polypeptide of the invention, included in the pharmaceutical preparations is such that a suitable dose within the designated range is provided (e.g., a dose within the range of 0.01-100 mg/kg of body weight).

The pharmaceutical composition for gene therapy can be in an acceptable diluent, or can include a slow release matrix in which the gene delivery vehicle is imbedded. If hydrodynamic injection is used as the delivery method, the pharmaceutical composition containing a nucleic acid molecule encoding a polypeptide described herein or a vector (e.g., a viral vector) containing the nucleic acid molecule is delivered rapidly in a large fluid volume intravenously. Vectors that may be used as in vivo gene delivery vehicle include, but are not limited to, retroviral vectors, adenoviral vectors, poxviral vectors (e.g., vaccinia viral vectors, such as Modified Vaccinia Ankara), adeno-associated viral vectors, and alphaviral vectors.

Pharmaceutical compositions that include the polypeptides of the invention as the therapeutic proteins may be formulated for, e.g., intravenous administration, parenteral administration, subcutaneous administration, intramuscular administration, intra-arterial administration, intrathecal administration, or intraperitoneal administration. The pharmaceutical composition may also be formulated for, or administered via, oral, nasal, spray, aerosol, rectal, or vaginal administration. For injectable formulations, various effective pharmaceutical carriers are known in the art. See, e.g., ASHP Handbook on Injectable Drugs, Toissel, 18th ed. (2014).

In some embodiments, a pharmaceutical composition that includes a nucleic acid molecule encoding a polypeptide of the invention or a vector containing such nucleic acid molecule may be administered by way of gene delivery. Methods of gene delivery are well-known to one of skill in the art. Vectors that may be used for in vivo gene delivery and expression include, but are not limited to, retroviral vectors, adenoviral vectors, poxviral vectors (e.g., vaccinia viral vectors, such as Modified Vaccinia Ankara (MVA)), adeno-associated viral vectors, and alphaviral vectors. In some embodiments, mRNA molecules encoding polypeptides of the invention may be administered directly to a subject.

In some embodiments of the present invention, nucleic acid molecules encoding a polypeptide described herein or vectors containing such nucleic acid molecules may be administered using a hydrodynamic injection platform. In the hydrodynamic injection method, a nucleic acid molecule encoding a polypeptide described herein is put under the control of a strong promoter in an engineered plasmid (e.g., a viral plasmid). The plasmid is often delivered rapidly in a large fluid volume intravenously. Hydrodynamic injection uses controlled hydrodynamic pressure in veins to enhance cell permeability such that the elevated pressure from the rapid injection of the large fluid volume results in fluid and plasmid extravasation from the vein. The expression of the nucleic acid molecule is driven primarily by the liver. In mice, hydrodynamic injection is often performed by injection of the plasmid into the tail vein. In certain embodiments, mRNA molecules encoding a polypeptide described herein may be administered using hydrodynamic injection.

The dosage of the pharmaceutical compositions of the invention depends on factors including the route of administration, the disease to be treated, and physical characteristics, e.g., age, weight, general health, of the subject. A pharmaceutical composition of the invention may include a dosage of a polypeptide of the invention ranging from 0.01 to 500 mg/kg (e.g., 0.01, 0.1, 0.2, 0.3, 0.4, 0.5, 1, 2, 3, 4, 5, 10, 15, 20, 25, 30, 35, 40, 45, 50, 100, 150, 200, 250, 300, 350, 400, 450, or 500 mg/kg) and, in a more specific embodiment, about 0.1 to about 30 mg/kg and, in a more specific embodiment, about 0.3 to about 30 mg/kg. The dosage may be adapted by the physician in accordance with conventional factors such as the extent of the disease and different parameters of the subject.

The pharmaceutical compositions are administered in a manner compatible with the dosage formulation and in such amount as is therapeutically effective to result in an improvement or remediation of the symptoms. The pharmaceutical compositions are administered in a variety of dosage forms, e.g., intravenous dosage forms, subcutaneous dosage forms, and oral dosage forms (e.g., ingestible solutions, drug release capsules). Generally, therapeutic proteins are dosed at 0.1-100 mg/kg, e.g., 1-50 mg/kg. Pharmaceutical compositions that include a polypeptide of the invention may be administered to a subject in need thereof, for example, one or more times (e.g., 1-10 times or more) daily, weekly, biweekly, monthly, bimonthly, quarterly, biannually, annually, or as medically necessary. In some embodiments, pharmaceutical compositions that include a polypeptide of the invention may be administered to a subject in need thereof weekly, biweekly, monthly, bimonthly, or quarterly. Dosages may be provided in either a single or multiple dosage regimens. The timing between administrations may decrease as the medical condition improves or increase as the health of the patient declines.

The invention is based on the discovery that substituting one or more specific amino acids from the human PAI-1 renders it capable of inhibiting NE activity and inhibiting NE activity wherein the NE is bound within NETs. These PAI-1 variant properties make for a useful therapeutic that can be used in the treatment of diseases characterized by aberrant NE activity and/or deficient A1AT activity.

In another aspect, the invention features a method of inhibiting NE activity in a subject in need thereof. In another aspect, the invention features a method of inhibiting NE activity bound in NETs in a subject in need thereof. The methods include administering to the subject a therapeutically effective amount of a polypeptide, nucleic acid molecule, or vector described herein or a pharmaceutical composition described herein.

In some embodiments of the method of inhibiting NE activity or NE activity bound within NETs in a subject, the subject has IPF and/or a condition characterized with aberrant NE activity (e.g., cystic fibrosis, chronic obstructive pulmonary disease (COPD), emphysema). In some embodiments of the method of inhibiting NE activity or NE activity bound within NETs in a subject, the subject has an A1AT activity and/or expression deficiency.

In another aspect, the invention features a method of treating a subject having IPF by administering to the subject a therapeutically effective amount of a polypeptide, nucleic acid molecule, or vector described herein or a pharmaceutical composition described herein.

In another aspect, the invention features a method of treating a subject having cystic fibrosis by administering to the subject a therapeutically effective amount of a polypeptide, nucleic acid molecule, or vector described herein or a pharmaceutical composition described herein.

In another aspect, the invention features a method of treating a subject having COPD by administering to the subject a therapeutically effective amount of a polypeptide, nucleic acid molecule, or vector described herein or a pharmaceutical composition described herein.

In another aspect, the invention features a method of treating a subject having emphysema by administering to the subject a therapeutically effective amount of a polypeptide, nucleic acid molecule, or vector described herein or a pharmaceutical composition described herein.

In another aspect, the invention features a method of treating a subject having acute respiratory distress syndrome (ARDS) by administering to the subject a therapeutically effective amount of a polypeptide, nucleic acid molecule, or vector described herein or a pharmaceutical composition described herein.

In another aspect, the invention features a method of treating a subject having ischemia reperfusion injury by administering to the subject a therapeutically effective amount of a polypeptide, nucleic acid molecule, or vector described herein or a pharmaceutical composition described herein.

In another aspect, the invention features a method of treating a subject having ethanol induced chronic pancreatitis by administering to the subject a therapeutically effective amount of a polypeptide, nucleic acid molecule, or vector described herein or a pharmaceutical composition described herein.

In another aspect, the invention features a method of treating a subject having rheumatoid arthritis (RA) by administering to the subject a therapeutically effective amount of a polypeptide, nucleic acid molecule, or vector described herein or a pharmaceutical composition described herein.

In another aspect, the invention features a method of treating a subject having disseminated intravascular coagulation (DIC) by administering to the subject a therapeutically effective amount of a polypeptide, nucleic acid molecule, or vector described herein or a pharmaceutical composition described herein.

In another aspect, the invention features a method of treating a subject having ulcerative colitis (UC) by administering to the subject a therapeutically effective amount of a polypeptide, nucleic acid molecule, or vector described herein or a pharmaceutical composition described herein.

In another aspect, the invention features a method of treating a subject having Crohn's disease by administering to the subject a therapeutically effective amount of a polypeptide, nucleic acid molecule, or vector described herein or a pharmaceutical composition described herein.

In another aspect, the invention features a method of treating a subject having dermatological diseases with neutrophil pathology by administering to the subject a therapeutically effective amount of a polypeptide, nucleic acid molecule, or vector described herein or a pharmaceutical composition described herein.

In another aspect, the invention features a method of treating a subject having an A1AT activity and/or expression deficiency by administering to the subject a therapeutically effective amount of a polypeptide, nucleic acid molecule, or vector described herein or a pharmaceutical composition described herein.

In another aspect, the invention features a method of treating a subject having any condition characterized with aberrant NE activity and/or expression by administering to the subject a therapeutically effective amount of a polypeptide, nucleic acid molecule, or vector described herein or a pharmaceutical composition described herein.

In another aspect, the invention features a method of treating a subject having any condition characterized with deficient A1AT activity and/or expression by administering to the subject a therapeutically effective amount of a polypeptide, nucleic acid molecule, or vector described herein or a pharmaceutical composition described herein.

In some embodiments of any of the above aspects, the subject has or is at risk of developing a condition characterized with aberrant NE activity (e.g., IPF, COPD, cystic fibrosis, emphysema).

In some embodiments of any of the above aspects, the subject has or is at risk of developing a condition characterized with deficient A1AT activity and/or expression.

One of ordinary skill in the art will readily recognize that the foregoing represents merely a detailed description of certain preferred embodiments of the present invention. Various modifications and alterations of the compositions and methods described above can readily be achieved using expertise available in the art and are within the scope of the invention.

### EXPERIMENTAL

### Example I

This example demonstrates that LMWuPA:PAI-1 complexes bind to LRP1 with higher affinity than PAI-1 alone.

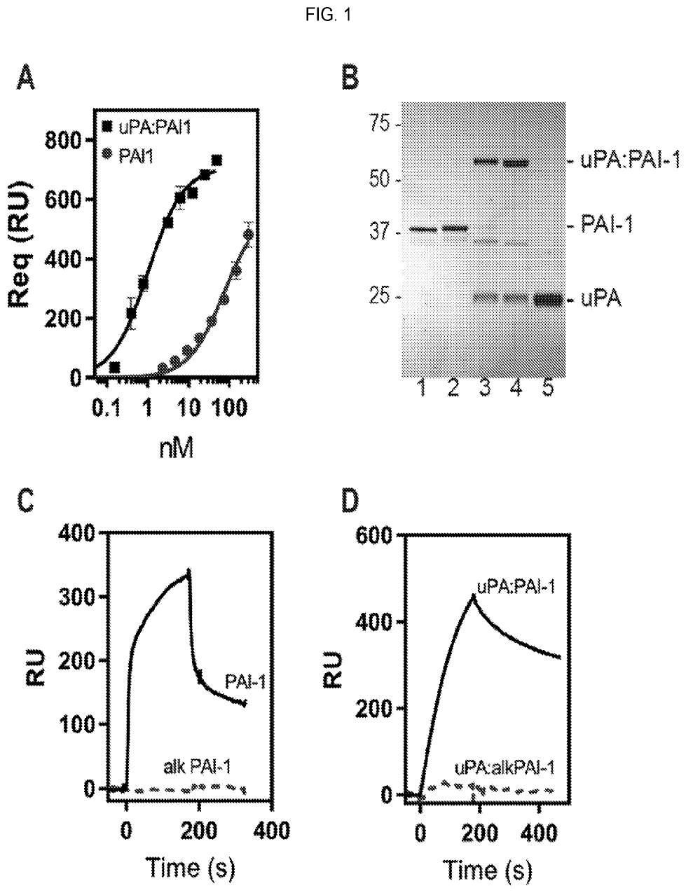

To resolve the conflict that exists in the literature regarding the relative affinities of PAI-1 and protease:PAI-1 complexes for LRP1, our initial experiment compared the binding of free PAI-1 and PAI-1 complexed to LMWuPA to LRP1. Notably, LMWuPA itself does not bind to LRP1. Since wild-type PAI-1 is relatively unstable and rapidly converts to the latent form, experiments in this and all subsequent studies (unless designated otherwise) used the 191L mutant of PAI-1 which extends the stability of PAI-1 from a half-life of 2.0 h to 18.4 h (see, Berkenpas, M. B., Lawrence, D. A., and Ginsburg, D. (1995) EMBO J. 14, 2969-77). Experiments were conducted that formed a complex of 191L PAI-1 with LMWuPA, and compared its binding with that of free PAI-1 to LRP1 using equilibrium SPR measurements. The data (FIG. 1A) reveal that the LMWuPA:PAI-1 complex binds almost 2 orders of magnitude tighter to LRP1 than PAI-1 alone (K_{D} for LMWuPA:PAI-1=0.9±0.2 nM while the K_{D} for PAI-1=74±13 nM).

### Example II

This example demonstrates that Lysine residues on PAI-1 are essential for binding of both PAI-1 and LMWuPA:PAI-1 complexes to LRP1.

To determine the contribution of lysine residues to the interaction of PAI-1 with LRP1 these residues were chemically modified with Sulfo-NHS-Acetate, which forms stable, covalent amide bonds with primary amines of lysine residues. This modification does not prevent PAI-1 from forming a stable complex with LMWuPA (FIG. 1B). Remarkably, however, this modification prevented the binding of both free PAI-1 (FIG. 1C) and the complex of LMWuPA with modified PAI-1 from binding to LRP1 (FIG. 1D). These results reveal that lysine residues on PAI-1 contribute to the binding of both PAI-1 and the LMWuPA:PA1-1 complex to LRP1.

### Example III

This example demonstrates that two charged residues are involved in the binding of PAI-1 to LRP1.

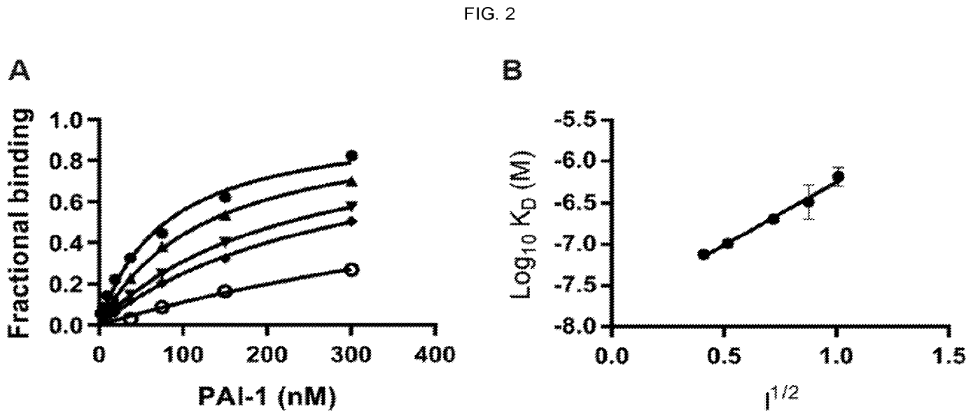

Experiments were next conducted that examined the effect of ionic strength on the binding of PAI-1 to LRP1. The results of these experiments reveal that the binding of PAI-1 to LRP1 is dependent upon ionic strength (FIG. 2A). FIG. 2B shows the data plotted in the form of a Debye-Hückel plot, where log₁₀ K_{D} is plotted versus ionic strength. These results suggest the involvement of 2 ionic interactions in the binding of PAI-1 with LRP1 as indicated by the slope (slope=1.5±0.1). This is consistent with the canonical model for the binding of ligands to LDL receptor family members in which two (or more) ε-amino groups of specific lysine residues located on the ligand form salt bridges with carboxylates of aspartate residues within the LDLa repeats (see, Fisher, C., et al., (2006) Mol. Cell. 22, 277-283).

### Example IV

This example demonstrates that kinetic analysis supports a bivalent model for binding of PAI-1 to LRP1.

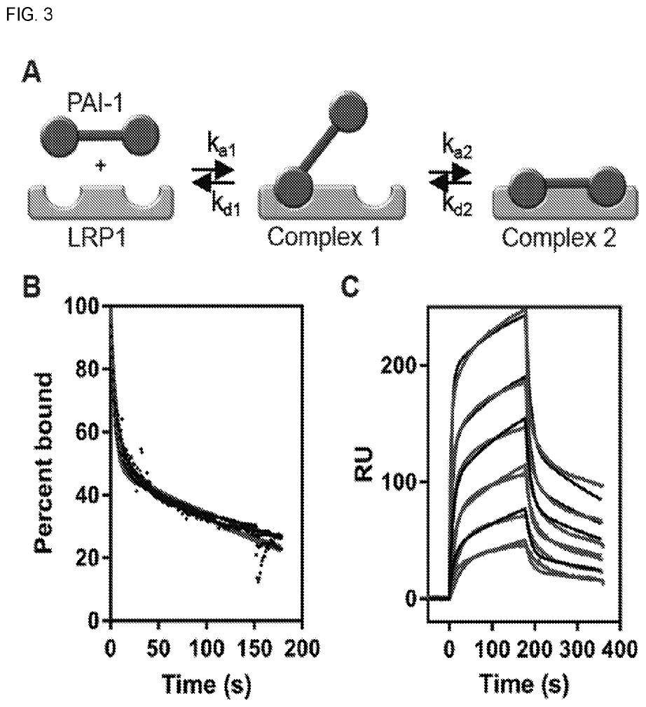

The data from FIG. 2 suggests the involvement of at least two charged residues in the interaction of PAI-1 with LRP1, raising the possibility of a bivalent binding model in which high affinity binding results from avidity effects mediated by interaction of two regions on PAI-1, each containing charged residues, with two LDLa repeats on LRP1 (FIG. 3A). A similar model has been proposed for the binding of FVIII to LRP1 (16). To test this model, kinetic measurements were performed examining the binding of I91L PAI-1 to LRP1 using surface plasmon resonance experiments. To gain insight into potential mechanisms, initially, the kinetics of PAI-1 dissociation from LRP1 were compared at various concentrations of ligand (FIG. 3B). These results reveal that dissociation kinetics occurs in two phases: a fast phase followed by a much slower phase. In addition, as expected for a bivalent model, the dissociation kinetics is independent of ligand concentration. The dissociation rate constants determined from a fit of the experimental data were used as initial estimates for the dissociation phase when the association and dissociation kinetics were simultaneously fit to a global bivalent model. This fit revealed that the experimental data are well described by a bivalent binding model (FIG. 3C). The kinetic data from the best fit (Table I) reveal a rapid association of PAI-1 with LRP1 to form complex I, and a conversion to complex II with a half-life of 97 s. Importantly, the value for the equilibrium binding constant, K_{D}, derived from kinetic analysis (65±6 nM) is close to the K_{D} value of 56±4 nM determined by equilibrium analysis of the SPR data. To determine if any difference occurs between the binding of I91L PAI-1 and wild-type PAI-1 to LRP1, we also investigated the detailed binding kinetics for wild-type PAI-1 with LRP1. The kinetic and equilibrium binding data are summarized in Table I and reveal kinetic constants and K_{D} values for wild-type PAI-1 that are similar to those for the I91L stable mutant.

TABLE I
Kinetic and equilibrium constants for the binding to WT PAI-1 and I91L PAI-1 to
LRP1

| Ligand/ | | | | | | ^{c}K_{D} |
|---|---|---|---|---|---|---|
| receptor | kₐ₁ (1/Ms) | k_{d1} (1/s) | kₐ₂ (1/s) | k_{d2} (1/s) | ^{b}K_{D} (nM) | (nM) |
| ^{d}WT PAI-1/ | 5.6 ± 0.7 X 10⁵ | 0.120 ± 0.004 | 1.1 ± 0.7 X 10⁻² | 5.1 ± 0.3 X 10⁻³ | 69 ± 3 | 85 ± 16 |
| LRP1 | | | | | | |
| ^{e}I91L PAI-1/ | 7.1 ± 0.4 X 10⁵ | 0.152 ± 0.007 | 7.1 ± 0.2 X 10⁻³ | 1.6 ± 0.4 X 10⁻³ | 65 ± 6 | 56 ± 4 |
| LRP1 | | | | | | |
| ^{d}I91L PAI-1/ | 5.1 ± 0.4 X 10⁵ | 0.097 ± 0.007 | 3.1 ± 0.2 X 10⁻³ | 1.3 ± 0.4 X 10⁻³ | 55 ± 5 | 49 ± 18 |
| Cluster IV | | | | | | |

^{a}Kinetic constants were obtained by fitting the data to a bivalent model.
^{b}The equilibrium binding constant K_{A} was calculated using the following equation:
K_{A} = (kₐ₁/k_{d1})*(1 + (kₐ₂/k_{d2}) and K_{D} was calculated as: K_{D} = 1/K_{A}
^{c}Calculated from equilibrium SPR measurements, in which Req was determined by the fitting the association data to a pseudo-first order process.
^{d}Three independent experiments were performed, and the values shown are the average ± SE.
^{e}Six independent experiments were performed, and the values shown are the average ± SE

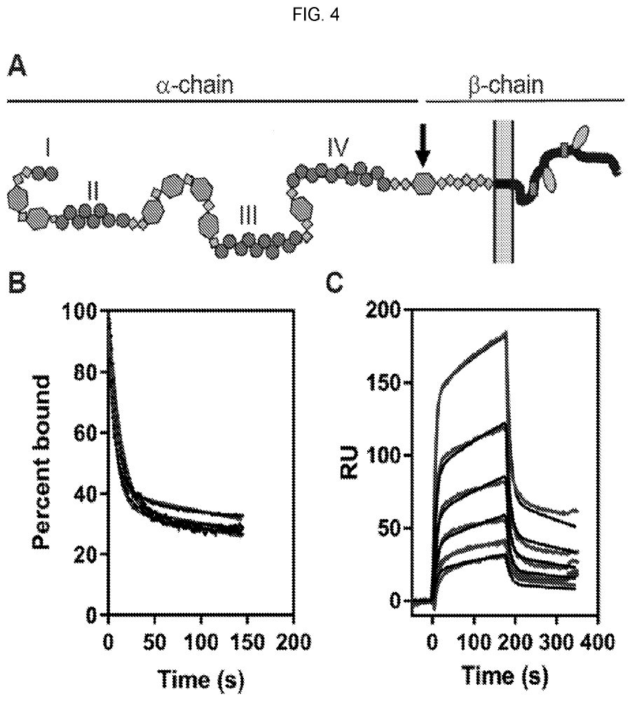

The ligand binding regions of LRP1 are mainly localized to clusters of LDLa repeats, termed clusters I, II, III and IV (FIG. 4A). Of these clusters, most ligands bind to clusters II, III or IV. Thus, experiments were conducted that also examined the binding of PAI-1 to clusters II, III and IV. Initial experiments revealed that 191L PAI-1 interacted with similar affinities to clusters II and IV, but with a much weaker affinity to cluster III. Detailed experiments were next conducted employing cluster IV, which is a major ligand binding region of LRP1. FIG. 4B confirms that the dissociation of PAI-1 from cluster IV also occurs with two phases and is independent of PAI-1 concentration. A comparison of the fit to experimental data reveal that the binding is consistent with a bivalent binding model (FIG. 4C). The best fit parameters are summarized in Table I, which reveal a K_{D} value of 55±5 nM derived from the kinetic data which is close to the K_{D} value of 49±18 nM estimated by equilibrium analysis of the SPR data. Thus, these results reveal that the binding of I91L PAI-1 to cluster IV is similar to its binding to full length LRP1.

### Example V

This example demonstrates a critical role for K207 in the binding of PAI-1 to LRP1.

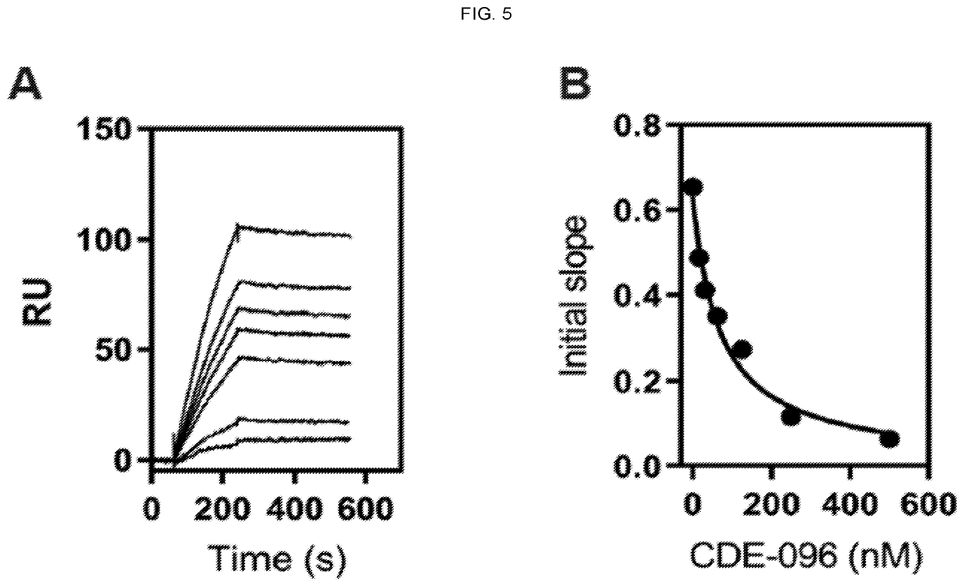

Since chemical modification of PAI-1 revealed a critical role for lysine residues in the interaction with LRP1, studies were initiated to identify specific lysine residues that contribute to the binding of PAI-1 to LRP1. To gain additional insight into regions on PAI-1 that may be important for its interaction with LRP1, the potential of CDE-096 to block the binding of HMWuPA:PAI-1 complexes to LRP1 was examined. CDE-096 is a small molecule inhibitor that binds reversibly to PAI-1 and inhibits the interaction of PAI-1 with proteases via an allosteric mechanism (see, Li, S.-H., et al., (2013) Proc. Natl. Acad. Sci. U.S.A. 110, E4941-9). CDE-096 also binds to uPA:PAI-1 complexes. When CDE-096 was added to HMWuPA:PAI-1 complexes, a dose-dependent inhibition of HMWuPA:PAI-1 binding to LRP1 was observed (FIG. 5A). An IC₅₀ of 70 nM was determined by re-plotting the initial slope of the association curve vs CDE-096 concentration (FIG. 5B).

Structural studies reveal that K207 and K263 contribute to the binding of CDE-096 with PAI-1 (see, Li, S.-H., et al., (2013) Proc. Natl. Acad. Sci. U.S.A 110, E4941-9). Thus, experiments were conducted that included these mutants of PAI-1 in the analysis, along with K69, K80 and K88 which have been previously identified as critical for the binding of PAI-1 to LDLa repeats from cluster II (see, Gettins, P. G. W., and Dolmer, K. (2016) J. Biol. Chem. 291, 800-812). In these studies, the K_{D} values were determined by SPR equilibrium measurements, and the data are summarized in Table II. The data demonstrate that mutation of K207 to alanine had the largest impact on PAI-1 binding to LRP1, decreasing the affinity by 19-fold. In addition, mutation of K69, K80 and K88 to alanine resulted in a 7-fold, 7-fold and 9-fold decrease in the affinity for LPR1, respectively. Interestingly, a PAI-1 molecule with mutations in both K80 as well as K207, or a mutant in which K80, K207 and K88 were all converted to alanine did not substantially reduce the affinity of binding over that of the K207A mutant alone and resulted in a 20-fold and 21-fold increase in K_{D}, respectively. The data in Table II also show the fractional surface area of the specific side chains based on the three-dimensional structure of PAI-1. The fractional accessible surface area of side chain groups is the area accessible to solvent in the protein divided by the calculated accessible surface area for that residue in and extended Gly-Xaa-Gly tripeptide (see, Willard, L., et al., (2003) Nucleic Acids Res. 31, 3316-3319) with values close to one being fully accessible and values close to zero being buried. The R76E PAI-1 mutant is deficient in LRP1 binding (see, Stefansson, S. (1998) J. Biol. Chem. 273, 6358-6366) and R76 has been proposed to be directly involved in the binding of PAI-1 to LRP1 (see, Gettins, P. G. W., and Dolmer, K. (2016) J. Biol. Chem. 291, 800-812). However, the ASA value of 0.24 reveals that this residue is buried and not available for direct interaction with LRP1. Likewise, K263 and K122, which has been implicated in prior studies, also are partially buried in the structure and therefore unavailable for direct interaction with LRP1.

TABLE II
^{a}Equilibrium binding constants for
mutant PAI-1 binding LRP1

| | | | | LMWuPA: | |
|---|---|---|---|---|---|
| | | PAI-1 | | PAI-1 | |
| | ^{b}Frac- | ^{c}Equi- | | ^{c}Equi- | |
| | tional | librium | | librium | |
| Mutation in | Surface | analysis | Fold | analysis | Fold |
| PAI-1 | ASA | K_{D} (nM) | change | K_{D} (nM) | change |
| I91L PAI-1 | | 56 | 1 | 0.5 | 1 |
| K263A | 0.38 | 180 | 3 | 0.7 | 1.4 |
| K176A | 0.43 | 189 | 3 | 0.7 | 1.4 |
| K122A | 0.2 | 229 | 4 | 1 | 2 |
| K69A | 0.95 | 388 | 7 | 1 | 2 |
| K80A | 0.6 | 397 | 7 | 2.9 | 5.8 |
| K88A | 0.61 | 508 | 9 | 0.7 | 1.4 |
| K207A | 0.68 | 1076 | 19 | 0.8 | 1.6 |
| 14-1B | 0.24 | 1774 | 32 | nd | |
| R76E PAI-1 | | | | | |
| K80/207A | | 1136 | 20 | 13 | 26 |
| K80/207/122A | | 477 | 9 | 25 | 50 |
| K69/80/207A | | 558 | 10 | nd | |
| K80/207/263A | | 862 | 15 | 16 | 32 |
| K80/207/88A | | 1184 | 21 | 122 | 244 |
| K80/207/176A | | 1206 | 22 | 7 | 14 |

^{a}Kinetic constants were obtained by fitting the data to a bivalent model. For each mutant, three independent experiments were performed, and the mean values are shown.
^{b}The fractional surface area was calculated using VADAR (Willard et al, Nucleic Acids Res 31, 3316 (2003) and 1dvm.pdb
^{c}Calculated from equilibrium measurements, in which Req was determined by the fitting the association data to a pseudo-first order process.

### Example VI

This example describes the binding LMWuPA:PAI-1 complexes to LRP1 occurs via complex mechanisms.

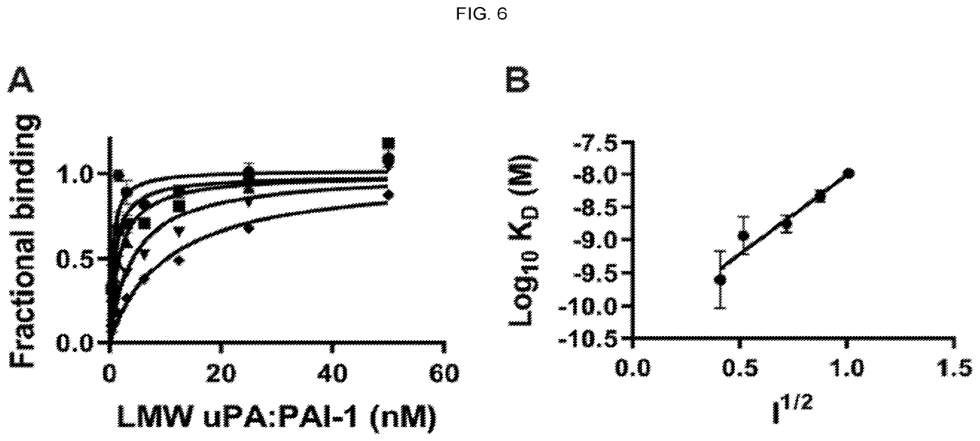

To characterize the binding of LMWuPA:PA1 to LRP1, experiments were conducted that examined the ionic strength dependence of the binding. The results shown in FIG. 6A demonstrate a significant dependence of binding upon the ionic strength. The Debye-Hückel plot (FIG. 6B) yields a slope of 2.4±0.3 suggesting the involvement of 2-3 ionic interactions in the binding.

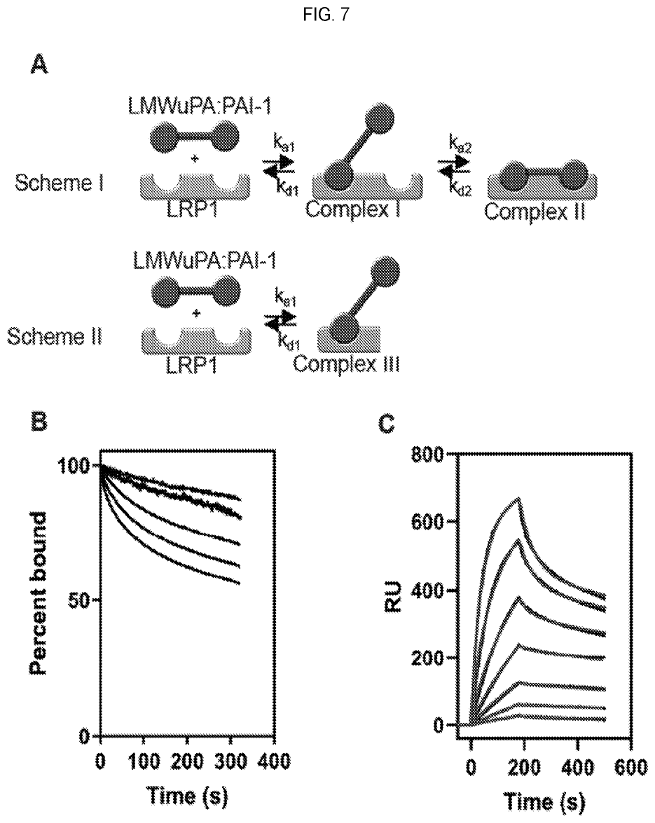

When the kinetics of the interaction were examined, it was observed that the dissociation kinetics changed at higher concentrations of LMWuPA:PAI-1 complexes (FIG. 7B). This is also apparent from the data in FIG. 7C, where a more rapid dissociation is noted at higher LMWuPA:PAI-1 concentrations which is suggestive of multiple binding mechanisms. This was observed before for the binding of RAP domains D1D2 to LRP1 (see, Prasad, J. M., et al., (2016) J. Biol. Chem. 291, 18430-18439). Thus, experiments also incorporated a second Scheme into the model in which LMWuPA:PAI-1 complexes are also able to bind to a second distinct site on LRP1 to form a monovalent complex (Complex III, FIG. 7A, Scheme II). To simplify the model, it was assumed that the kₐ₁ and k_{d1} in Scheme II is identical to kₐ₁ and k_{d1} in the first step in Scheme I. When the experimental SPR data were fit to a model containing both Scheme I and Scheme II, an excellent fit was obtained (FIG. 7C) and the kinetic parameters are summarized in Table III. The data confirm high affinity binding of LMWuPA:PAI-1 complex to LRP1 with a K_{D} that is ˜100-fold greater than that of PAI-1 alone, mostly attributable to slower dissociation rates for LMWuPA:PAI-1 complexes (compare k_{d1} and k_{d2} for PAI-1 in Table I with those values for LMWuPA:PAI-1 complexes in Table III).

^{a}TABLE III
Kinetic and equilibrium binding constants for the binding to LMWuPA:I91L PAI-1 and
mutants to Cluster IV and to LRP1

| Receptor/ | | | | | | K_{D2} |
|---|---|---|---|---|---|---|
| fragment | kₐ₁ (1/Ms) | k_{d1} (1/s) | kₐ₂ (1/s) | k_{d2} (1/s) | K_{D1} (nM) | (nM) |
| ^{b}LRP1 | 9.1 ± 0.4 X 10⁵ | 1.8 ± 0.2 X 10⁻² | 4.2 ± 0.9 X 10⁻² | 1.8 ± 0.6 X 10⁻³ | 0.8 ± 0.2 | 21 ± 3 |
| ^{b}Cluster IV of | 1.3 ± 0.5 X 10⁶ | 4.6 ± 0.3 X 10⁻² | 1.4 ± 1.3 X 10⁻¹ | 1.0 ± 0.6 X 10⁻² | 2.7 ± 0.5 | 37 ± 9 |
| LRP1 | | | | | | |

^{a}Kinetic constants were obtained by fitting the data to a bivalent model (FIG. 5A).
^{b}Three independent experiments were performed, and the values shown are the average ± SD.

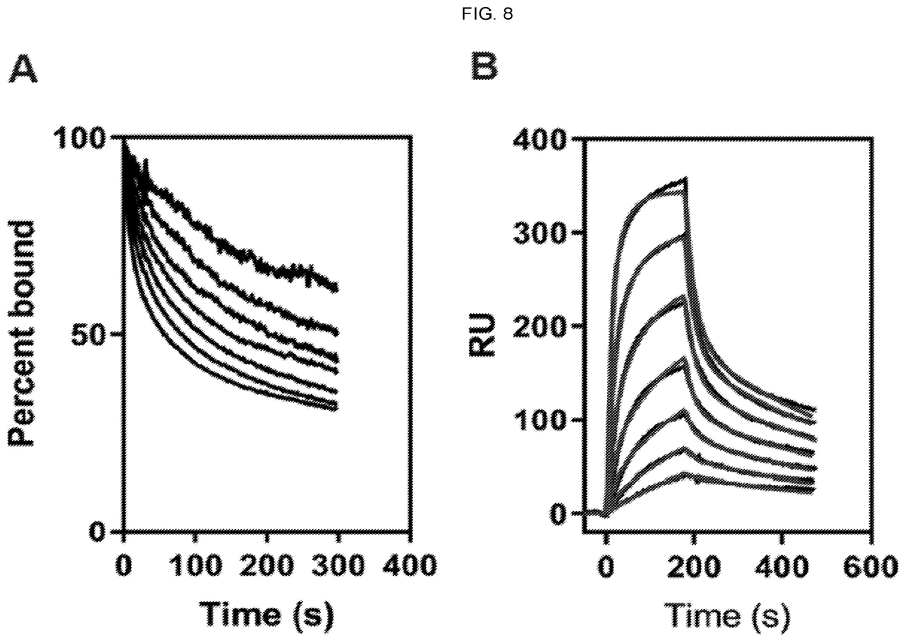

Experiments also examined the binding of LMWuPA:PAI-1 complexes to cluster IV of LRP1 immobilized on the SPR chip. Similar to the binding of LMWuPA:PAI-1 to full length LRP1, the dissociation kinetics was not independent of ligand concentration (FIG. 8A), and thus experiments fit the data to the model described in FIG. 7A. The data was well described by the fit (FIG. 8B), and the parameters derived from these fits are summarized in Table III and reveal that LMWuPA:PAI-1 complexes bind slightly weaker to Cluster IV than to full length LRP1. These results suggest that LMWuPA:PAI-1 complexes may also interact with regions of LRP1 that are outside of Cluster IV.

### Example VII

This example demonstrates that PA1-I mutants reveal additional residues are involved in the interaction of LMWuPA:PAI-1 complexes with LRP1.

To determine if similar lysine residues on PAI-1 are also involved in the interaction of uPA:PAI-1 complexes with LRP1, experiments were conducted that also formed complexes of LMWuPA with mutant PAI-1 molecules and measured the binding of these complexes to LRP1. The results of these studies are summarized in Table II. Interestingly, unlike the binding of PAI-1 to LRP1, individual mutants of PAI-1 (K69A, K88A and K207A) had minimal impact on the binding of LMWuPA:PAI-1 complexes to LRP1 while K80A resulted in a 5.8-fold decrease in K_{D}. A PAI-1 molecule containing a double mutant of K80A and K207A resulted in a 23-fold decrease in the binding of LMWuPA:PAI-1 complex to LRP1. Strikingly, the triple mutant of K80A, K207A and K88A resulted in a 244-fold decrease in affinity (Table II) revealing a critical role for these three residues in the LMWuPA:PAI-1 complex for binding to LRP1.

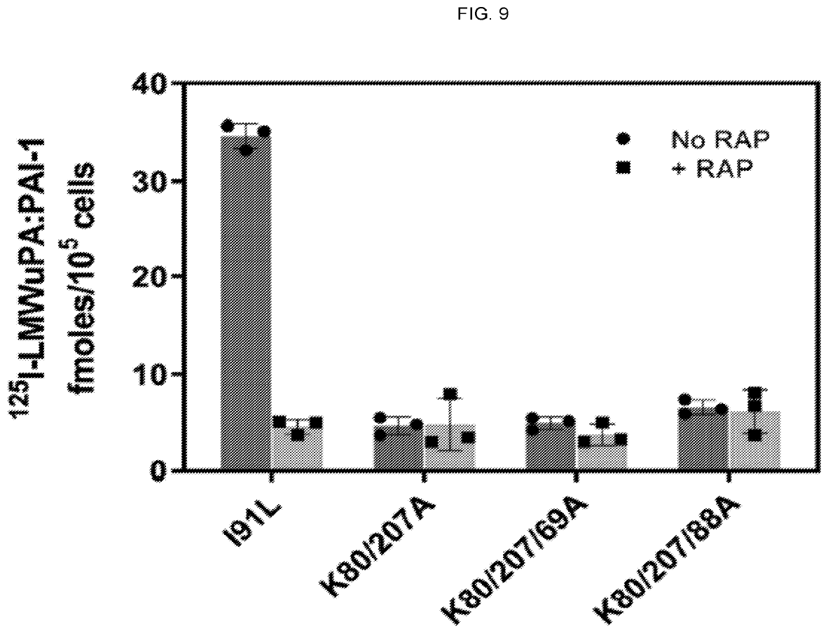

Experiments next examined the cellular uptake of complexes of PAI-1 with LMWuPA formed from PAI-1 molecules containing double or triple mutations (FIG. 9). The results reveal when complexed to LMWuPA, the double and triple mutants of PAI-1 were not effectively taken up by cells expressing LRP1.

### Example VIII

This example describes the materials and methods utilized for Examples I-VII.

### Reagents.

LMWuPA, HMWuPA, WT PAI-1 HMWuPA:PAI-1 complexes and I91L PAI-1 were purchased from Molecular Innovations. Mutant PAI-1 proteins were produced and purified as described. LRP1 was purified from human placenta as described (see, Ashcom, J. D., et al., (1990) J. Cell Biol. 110, 1041-1048). LRP1 ligand binding clusters II, III and IV were purchased from RnD Systems. CDE096 was synthesized as described (see, Li, S.-H., et al., (2013) Proc. Natl. Acad. Sci. U.S.A 110, E4941-9). LMWuPA:PAI-1 complexes used for Biacore studies were formed by incubating PAI-1 with 1.2 fold molar excess of LMWuPA in PBS for 1 h at room temperature. Complex formation was verified by analyzing proteins on 4-20% Tris-gly gel (Novex) and staining with colloidal blue stain.

### Chemical Modification of I91L PAI-1.

Chemical modification of I91L PAI-1 to block the primary amines in lysine side chains was performed using Sulfo-NHS-acetate (Thermo-Fisher Scientific). Sulfo-NHS-acetate was dissolved in PBS at 50 mg/ml. 55 ug of I91L PAI1 was incubated with 50-fold excess of the Sulfo-NHS-acetate over total amino groups in I91L PAI-1 in PBS for 3 h at 4 degrees centigrade. The modified I91L PAI-1 protein was desalted into 0.01M HEPES, 0.15MNaCl pH7.4, using NAP™-5 Sephadex G-25 column (GE Healthcare) to remove the excess Sulfo-NHS-acetate.

### Surface Plasmon Resonance.

Purified LRP1 was immobilized on a CMS sensor chip surface to the level of 10,000 response units, using a working solution of 20 μg/ml LRP1 in 10 mM sodium acetate, pH 4. LRP1 ligand binding cluster IV was immobilized on a CMS sensor chip surface to the level of 2,000 response units, using a working solution of 20 μg/ml cluster IV in 10 mM sodium acetate, pH 4 according to the manufacturer's instructions (BIAcore AB). An additional flow cell was activated and blocked with 1 M ethanolamine without protein to act as a control surface. Unless otherwise stated binding experiments were performed in HBS-P buffer (0.01 M HEPES, 0.15 M NaCl, 0.005% surfactant P, 1 mM CaCl2, pH 7.4). For ionic strength dependency buffers were made with 10 mM HEPES, 0.0005% surfactant P, 1 mM CaCl2 with various concentrations of NaCl (0.15 M, 0.25 M, 0.5 M, 0.75 M and 1.0 M), pH7.4. All experiments were performed on a BIAcore 3000 instrument, using a flow rate of 20 μl/min at 25° C. Sensor chip surfaces were regenerated by 15-s injections of 100 mM phosphoric acid at a flow rate of 100 μl/min.

### SPR Data Analysis.

Dissociation rates were fit to a 2-expential decay using GraphPad Prism 7.04 software. Kinetic data were analyzed to a bivalent model (Scheme 1) using numerical integration algorithms available in BIAevaluation shotware:

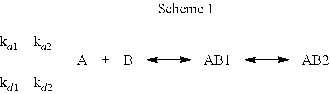

Where A represents ligand (PAI-1 or LMWuPA:PAI-1 complex), B represents LRP1, AB1 represents ligand:LRP1 complex at site 1 and AB2 represents ligand:LRP1 complex at site 2. To facilitate the fitting process, estimates for k_{d1} and k_{d2} were obtained by fitting the dissociation data globally to a two exponential decay model. These values were then used as initial estimates in the fitting process. In the case of LMWuPA:PAI-1 complexes, the data were fit to the following Schemes as previously described:

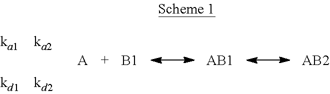

### And

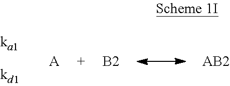

Equilibrium binding data was determined by fitting the association rates to a pseudo-first order process to obtain Req. Req was then plotted against total ligand concentration and fit to a binding isotherm using non-linear regression analysis in GraphPad Prism 7.04 software:

y=B max*L/(KD+L)

Where Bmax is the Req value at saturation, L is the free ligand concentration and K_{D} is the equilibrium binding constant. Since the free ligand concentration is unknown in these experiments, the use of this equation assumes that the total amount of added ligand is far greater than the amount of ligand bound to the LRP-1 coupled SPR chip.

### Inhibition by CDE-096.

HMWuPA-PAI-1 complex was diluted to 2 nM in 0.01 M HEPES, 0.15 M NaCl, 1 mM CaCl2, 0.0005% surfactant P, 0.1% DMSO, pH 7.8 containing 0 to 500 nM CDE-096. Binding to LRP1 on Biacore was done as above except running buffer was 0.01 M HEPES, 0.15 M NaCl, 1 mM CaCl2, 0.0005% surfactant P, 0.1% DMSO, pH7.8.

### Uptake of LMWuPA PAI-1 Complexes by Cells.

WI38 cells were plated in 12 well tissue culture plates previously coated with poly-D-lysine hydrobromide (Sigma). Cells were incubated in assay buffer (DMEM, 1% BSA, 20 mM HEPES) for 1 h before treating with Iodinated complex. LMWuPA was iodinated with I-125 sodium iodide (Perkin Elmer NEZ033) using Iodo-gen (Pierce) in PBS containing 1 mM 6-aminocaproic acid (Aldrich). Iodinated protein was desalted into PBS using PD-10 column (GE Healthcare) to remove free iodine. Labeled complex was formed by incubating I91L PAI-1 and its mutants (0.8 uM) with I-125 LMWuPA (0.4 uM) for one hour at room temperature. Resulting complex was diluted to 5 nM in assay buffer alone or assay buffer containing 1 uM RAP and placed on cells for 6 h at 37 degrees. Media was removed, cells were washed with 2 ml PBS and treated with trypsin (Corning 25-0520) containing 50 ug/ml proteinase K. Cells were centrifuged at 4000 rpm for 4 min. Supernatant was removed and the cell pellet counted to determine moles internalized.

### Example IX

This example describes the purification of a PAI-1 variant having the following mutations within wild-type human mature PAI-1 amino acid sequence (SEQ ID NO: 3): R101A and Q123K (hereinafter, “MDI-1003) in E. coli.

Two hundred ml of MDI-1003 fermentor lystate centrifugally clarified supernate was dialyzed against 0.05 M Sodium Phosphate, 0.1 M Sodium Chloride, 0.001 M EDTA, pH 6.6, then chromatographed on a Heparin-Sepharose 6B column (10×5.0 cm) at a flow rate of approximately 0.5 ml/min at room temperature. The Heparin-Sepharose 6B column was washed with 2 L of 0.05 M Sodium Phosphate, 0.1 M Sodium Chloride, 0.001 M EDTA, pH 6.6, followed by a 600 ml gradient elution to 1.0 M Sodium Chloride in the same buffer. PAI-1 containing fractions were pooled and solid Ammonium Sulfate was added to 18% saturation. The PAI-1 was chromatographed on a Phenyl-Sepharose Fast Flow (Low Sub) column (10×2.5 cm) previously equilibrated in 0.05 M Potassium Phosphate, 0.1 M Sodium Chloride, 0.001 M EDTA, pH 6.6, 30% saturated Ammonium Sulfate, at a flow rate of approximately 0.5 ml/min at room temperature. The Phenyl-Sepharose Fast Flow column was washed with 500 ml of 0.05 M Potassium Phosphate, 0.1 M Sodium Chloride, 0.001 M EDTA, pH 6.6, 30% saturated Ammonium Sulfate, followed by a 400 ml gradient elution to 0% Ammonium Sulfate in the same buffer. PAI-1 containing fractions were pooled and precipitated by addition of solid Ammonium Sulfate to 65% saturation. The precipitate was dissolved to 3.5 mg/ml with 0.05 M Sodium Phosphate, 0.1 M Sodium Chloride, 0.001 M EDTA, pH 6.6, then extensively dialyzed against the same buffer. The yield after the Heparin-Sepharose 6B column was 90 ml containing 80 mg protein (lane 1). The yield after the Phenyl-Sepharose Fast Flow column was 80 ml containing 47 mg protein (lane 2). The final yield was 12 ml containing 38 mg highly purified HPAI-AVI-AK protein.

### Example X

### CF-Sputum Titration for Elastase Concentration.

Sputum from CF patient is extracted in 2 mL of cold PBS/1 g sputum, then hand homogenize until smooth. Centrifuge at 10,000×g for 20 min (4° C.) and save sup for elastase titration. Next dilute purified HNE to 40 nM and add 0, 2.5, 5, 7.5, 10, 12.5, 15, and 20 uL to each well (black plate) and bring volume to 100 uL with 40 mM Hepes, 100 mM NaCl, pH7.4, 0.005% Tween-20. Add 100 uL of 500 uM MeOSuc-AAPV-AMC and read kinetically 10 min ex 370 em 440. Note slope and intercept.

Dilute CF-sputum and add 0, 2.5, 5, 7.5, 10, 12.5, 15 and 20 uL to each well, bring volume to 100 uL with 40 mM Hepes, 100 mM NaCL, pH7.4, 0.005% Tween-20 and add 100 uL 500 uM MeOSuc-AAPV-AMC and read kinetically 10 min ex370, em440. Back calculate the elastase concentration in the CF-sputum using the slope and intercept of the purified FINE.

### IC₅₀/Kinetics.

Incubate 50 nM HNE or CF-sputum+/−salmon sperm DNA or heparin for 30 min at room temp. Serial dilute elastase inhibitor into black plate (diluted in 40 mM Hepes, 100 mM NaCl, pH7.4, 0.005% Tween-20) to a final volume of 90 uL. Add 10 uL of the HNE/DNA/Heparin from above to the 90 uL of inhibitor. Incubate at room temp. for 30 sec, 1 min, 2 min, or longer if needed. Add 100 uL of 500 uM MeOSucAAPV-AMC to each well and read kinetically 10 min ex370, em440.

eKinetics Calculations

K_{obs}=Ln(nM elastase remaining/nM elastase starting))/time (sec)

K_{obs} plotted against inhibitor concentration and Vₘₐₓ and Kₘ determined by non-linear fit michaelis-menten.

Kᵢ=Vₘₐₓ/Kₘ

### PK Study of AVI and AVI-AK

Pharmacokinetics characteristics of AVI or AVI-AK were assessed following an intravenous (IV) bolus administration at 20 mg/kg in 8 week old C57BL/6J male mice. Blood was drawn 0.5, 1, 2, 6 and 24h later and the amount of PAI-1 in plasma was determined.

### Acute Lung Edema Via LPS Injection

Wild type C57BL/6J mice (male 8 weeks old) were subjected to intratracheal instillation of LPS (25 microL at 2 mg/mL), followed with intratracheal treatment of vehicle, AVI, AVI-AK, or Aralast (30 microL at 1.3 mg/mL). Eighteen hours later, animals were PBS perfused and wet lung weights and total elastase were obtained.

### Lung Extraction and Homogenization

Get whole lung wet weight. Add 250 uL of 0.4M Hepes, 0.1M NaCl, pH7.4, 1% Tx-100 and grind with homogenizer at high speed for 1 min. Centrifuge for 10 min at 10,000×g (4° C.). Remove sup to new tube and re-spin 10,000×g 10 min. Remove sup to new tube for assays.

### Fibrosis Assays

Fibrosis assay were essentially as described (Blood, 2011, 118:2313-2321). Briefly, Weight- and age-matched (18-22 g at 6-8 weeks of age) WT mice were treated on day 0 with a single dose of intratracheal bleomycin (1.15 u/kg in 50 L of sterile PBS) to induce lung fibrosis. Starting on Day 1 mice are administered either MDI-1001, MDI-1002, MDI-1003 or saline twice daily (4 mg/kg IP) to treat the acute injury phase. At Day 21 mice are sacrificed and lung fibrosis determined from hydroxyproline measurements as described (see, Blood, 2011, 118:2313-2321).

### Construction of HFc-AVI-AK Expression Vector and Transfected CHO Cell Line

The following mutations were introduced into the mature form of the human PAI-1 cDNA by site-directed mutagenesis (QuickChange II Kit, Agilent, Santa Clara, Calif.): I91L, R101A, Q123K, V343A, R346V. Mutations V343A, R346V, I91L (AVI) convey a stable active PAI-1 phenotype, whereas mutations R101A, Q123K (AK) introduce a reduced vitronectin-binding phenotype. The modified cDNA (designated AVI-AK) was cloned into the pcDNA5/FRT plasmid with an N-terminal fusion peptide sequence consisting of a human immunoglobin (IgG1) constant region comprised of domains 2 and 3, including hinge region sequences (HFc). Integrity of the construct was confirmed by restriction digest screening and DNA sequencing. The verified HFc-AVI-AK fusion plasmid was co-transfected using GeneJuice reagent (Novagen/Millipore) into CHO (Chinese hamster ovary) Flp-In cells (InVitrogen) with the pOG44 plasmid (bearing the Flp recombinase gene) at a 9:1 ratio to facilitate single-copy integration of the fusion protein sequence. The transfected cells were incubated for 48 hours at 37° C., 6% CO2 prior to addition of 100 ug/ml hygromicin (Invivogen) to select for cells bearing the integrated AVI-AK cDNA. Following selection and propagation of the transfected cells in Ham's F12 media (supplemented with 10% fetal bovine serum and hygromycin), the cell culture was adapted to growth in serum-free media (CHOgro, Mirus Bio) to facilitate non-adherent growth, amplify cell density, and simplify purification. Protein expression is regulated by a constitutive cytomegalovirus (CMV) promotor, so cell supernatant media containing HFc-AVI-AK was harvested approximately every 3-5 days for downstream processing.

### Purification of HFc-AVI-AK

Conditioned media was diluted 1:1 with phosphate buffered saline (PBS, pH 7.0) and applied to a column containing 15-20 ml Protein A/Protein G resin equilibrated in PBS, then extensively washed with PBS. Bound fusion protein was eluted with 0.1M glycine, 0.1M NaCl, pH 3.0 and collected into 0.5M sodium acetate pH 5.6 to stabilize the pH, yielding >95% pure protein. The protein eluate was then immediately applied to heparin sepharose equilibrated in 0.05M sodium phosphate, 0.1M NaCl, pH 6.6 then washed and eluted with 0.05M sodium phosphate, 1M NaCl, pH 6.6. This second step takes advantage of the unique properties of PAI-1 to concentrate the protein and achieve >99% purity.

### Results

MDI-1001— PAI-1 variant having the following stabilizing mutation within wild-type mature PAI-1 amino acid sequence (SEQ ID NO: 3): I91L and the NE inhibition enabling mutations V343A and R346V
MDI-1002— Fc fusion protein with MDI-1001 mutations
MDI-1003— PAI-1 variant of MDI-1001 having the additional mutations within wild-type human mature PAI-1 amino acid sequence that disable the vitronectin binding function of PAI-1 (SEQ ID NO: 3): R101A and Q123K
MDI-1004— Fc fusion protein with MDI-1003 mutations

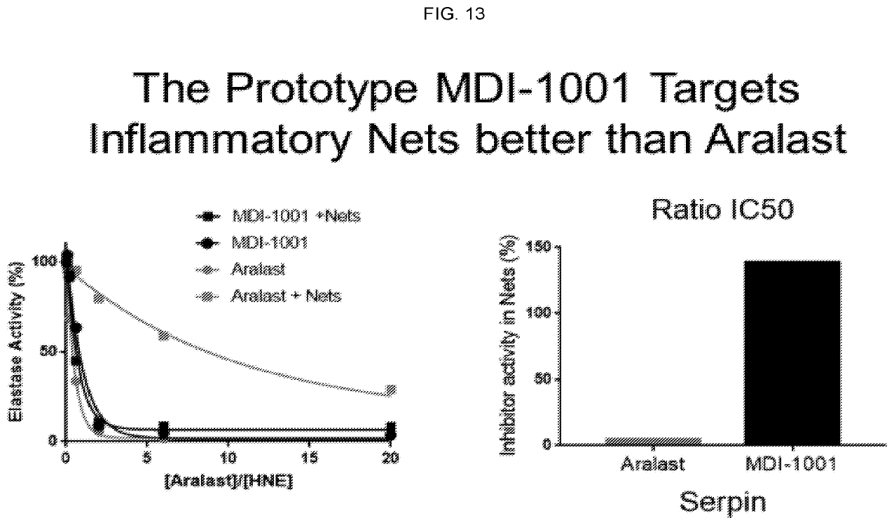

FIG. 13 shows that MDI-1001 targets inflammatory nets better than Aralast.

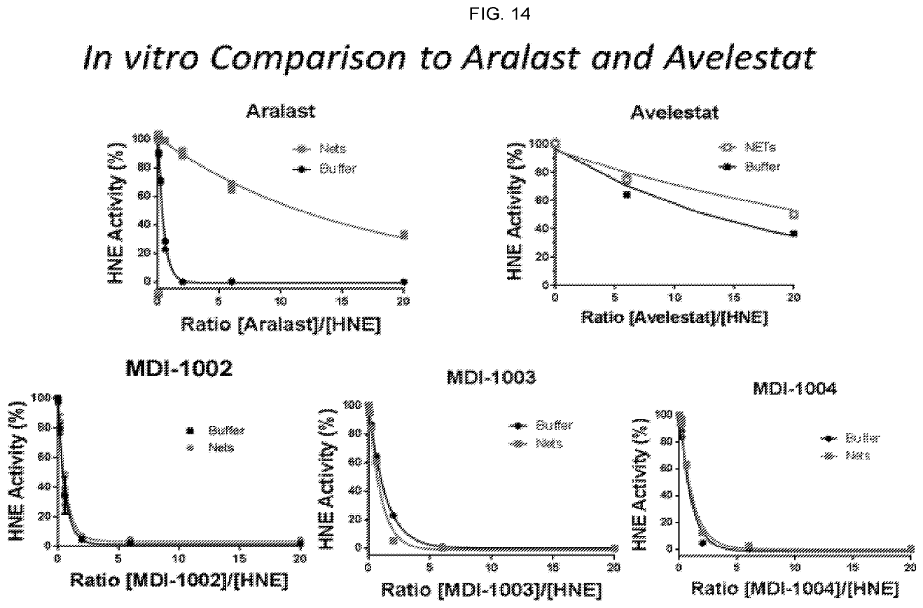

FIG. 14 shows an in vitro comparison between Aralast, Avelestat, MDI-1002, MDI-1003, and MDI-1004.

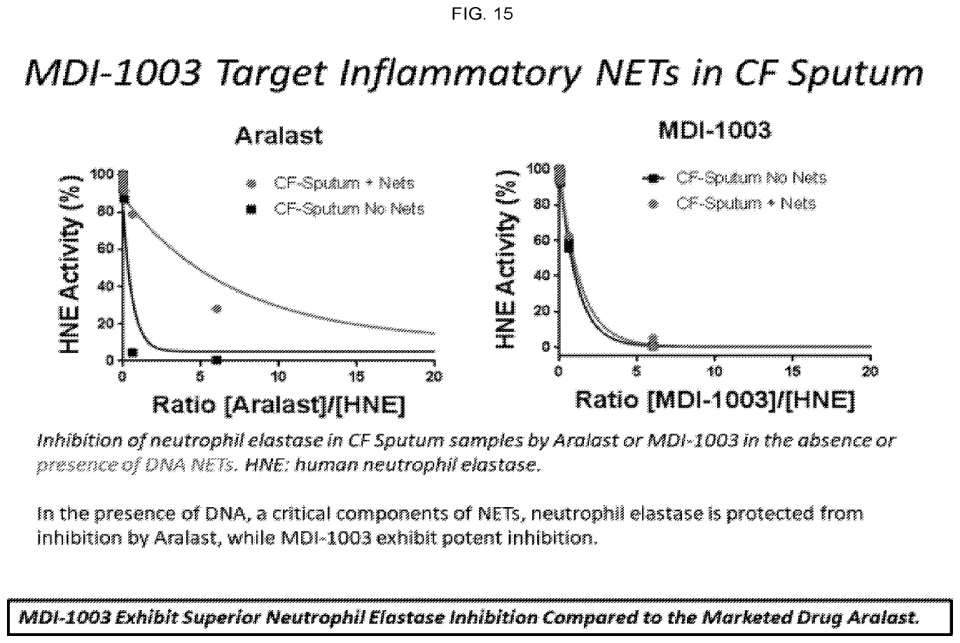

FIG. 15 shows that MDI-1003 targets NETs in CF sputum.

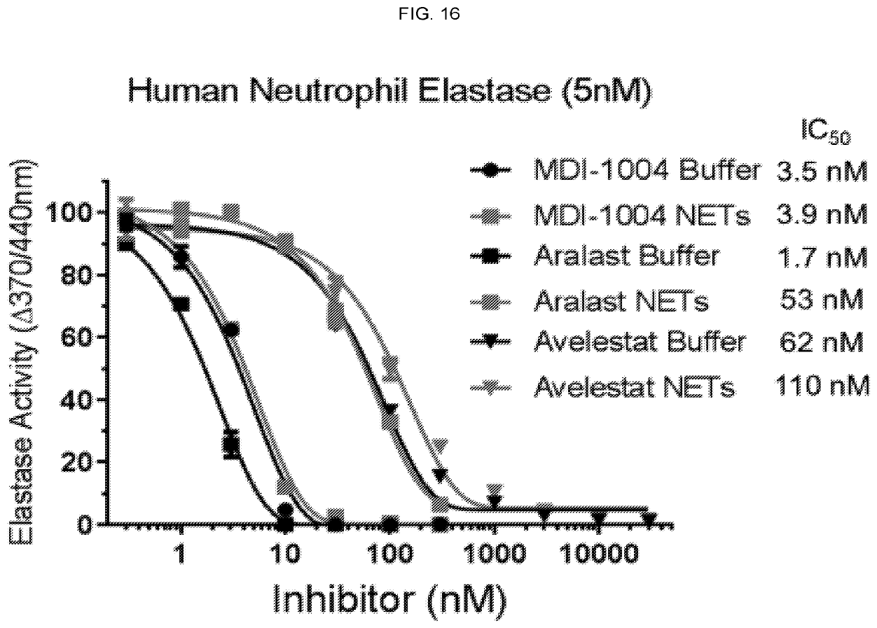

FIG. 16 shows elastase activity as a function of inhibitor concentration.

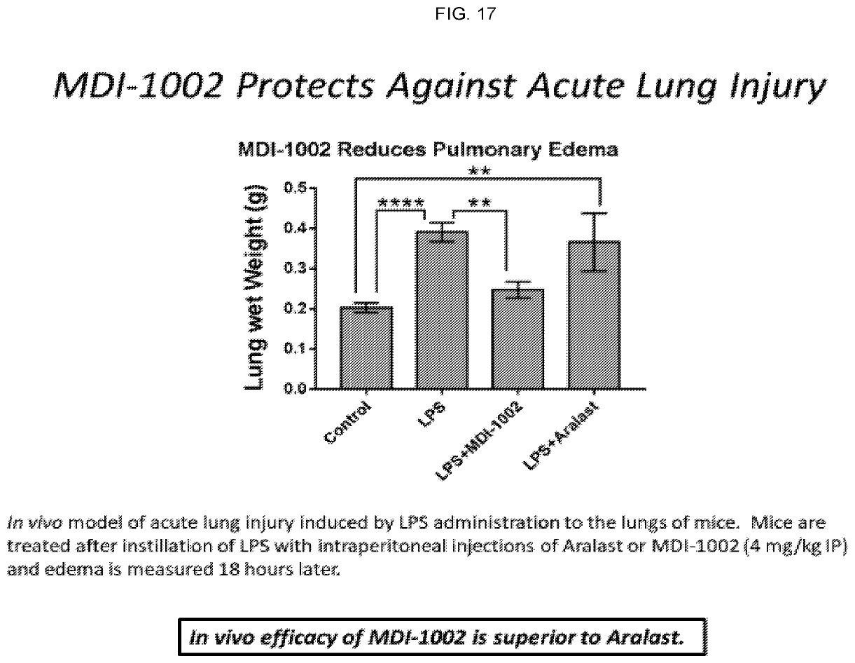

FIG. 17 shows that MDI-1002 protects against acute lung injury.

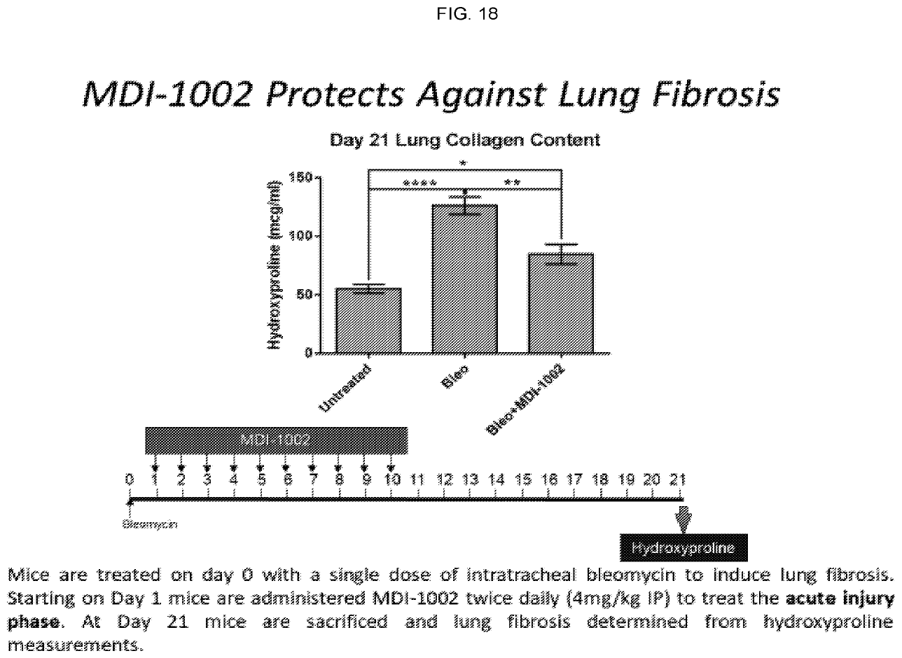

FIG. 18 shows that MDI-1002 protects against lung fibrosis.

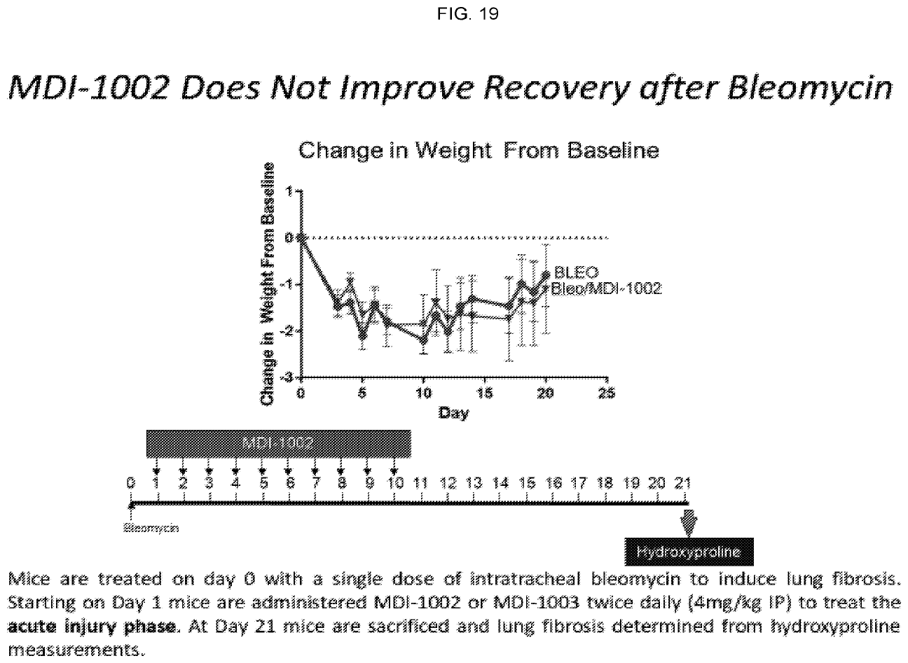

FIG. 19 shows that MDI-1002 does not improve recovery after bleomycin.

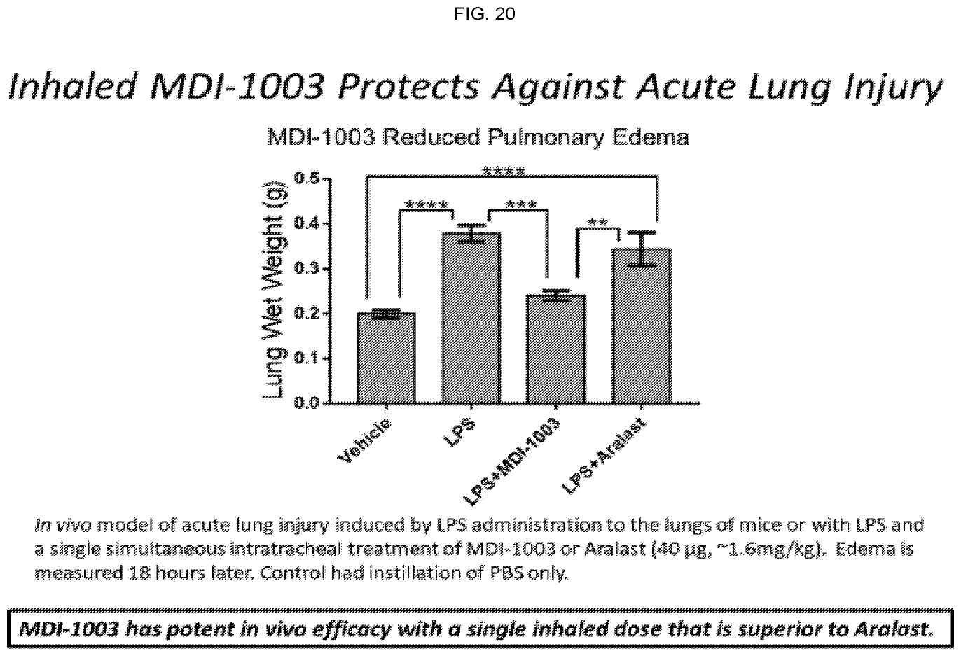

FIG. 20 shows that inhaled MDI-1003 protects against acute lung injury.

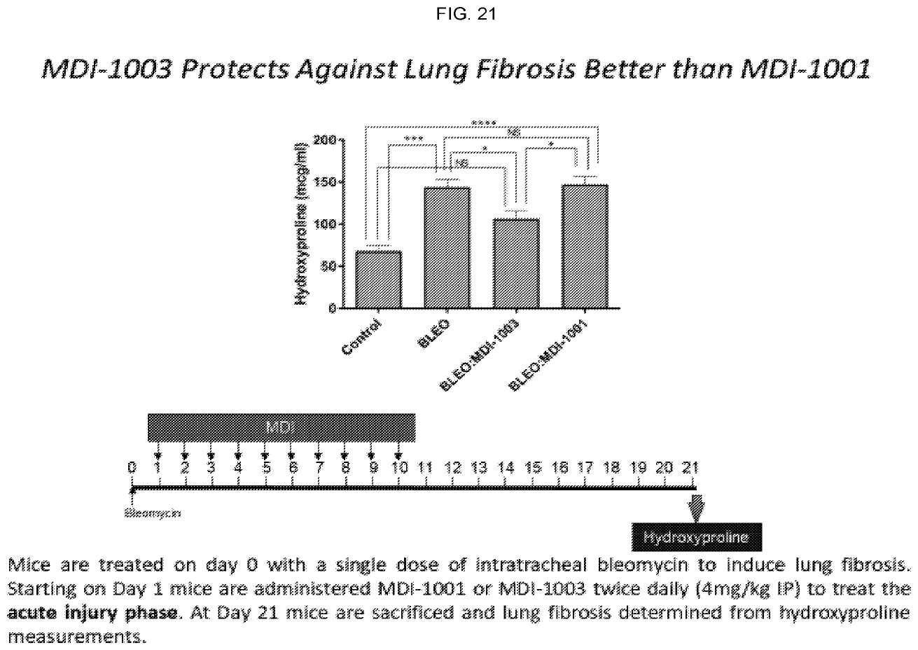

FIG. 21 shows that MDI-1003 protects against lung fibrosis better than MDI-1001.

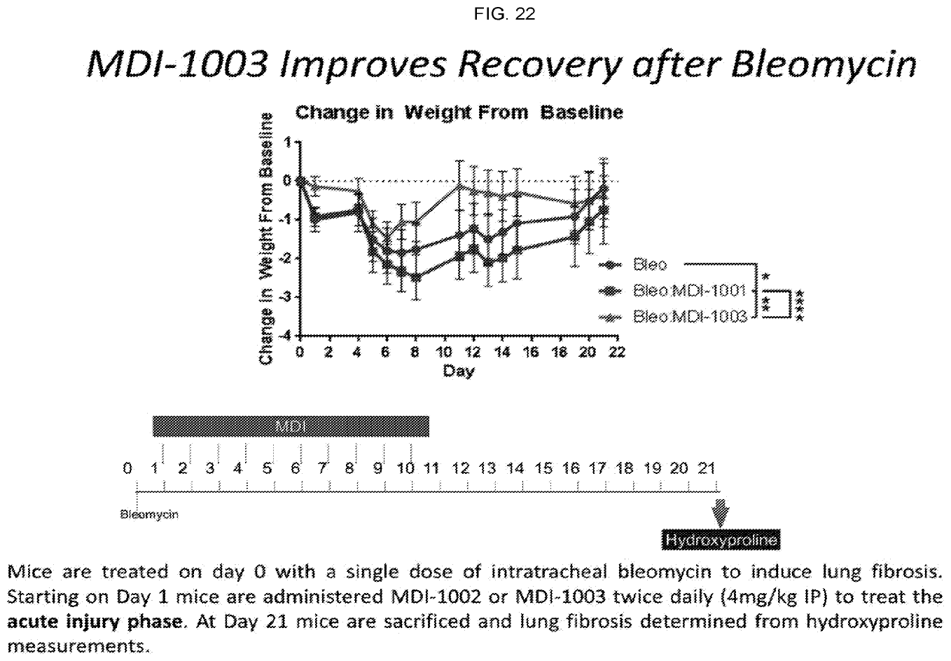

FIG. 22 shows that MDI-1003 improves recovery after bleomycin.

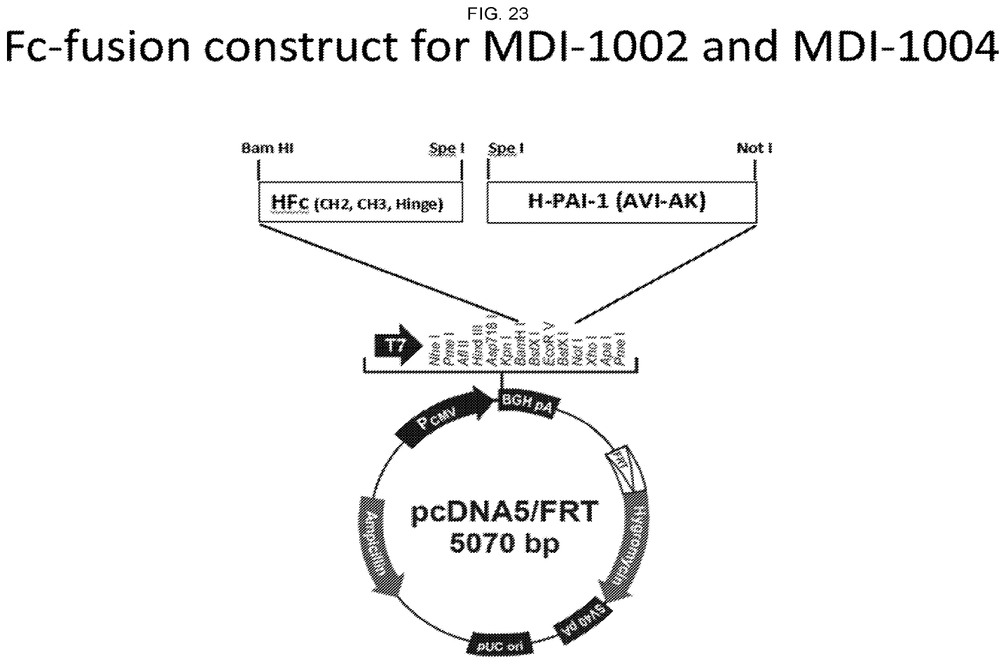

FIG. 23 shows an Fc-fusion construct for MDI-1002 and MDI-1004.

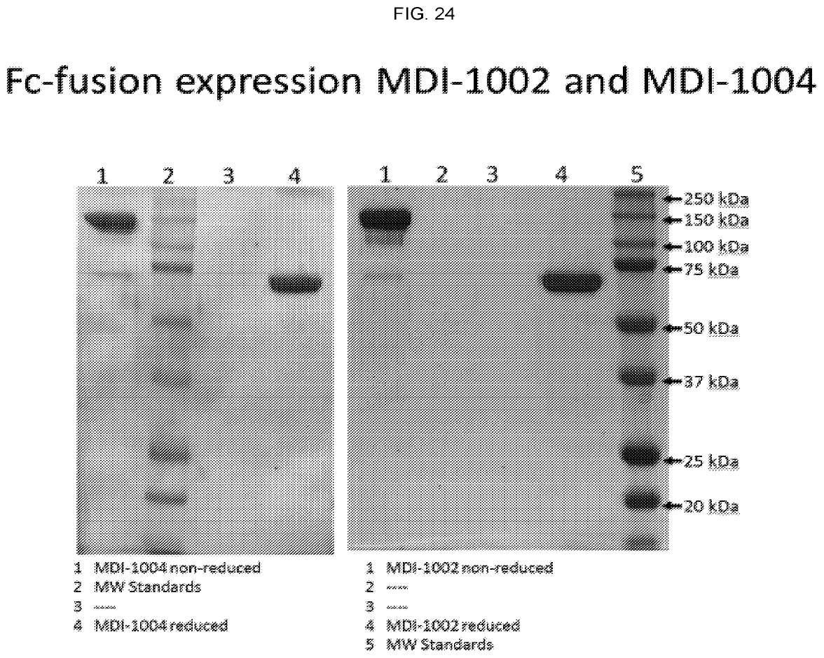

FIG. 24 shows Fc-fusion expression of MDI-1002 and MDI-1004.

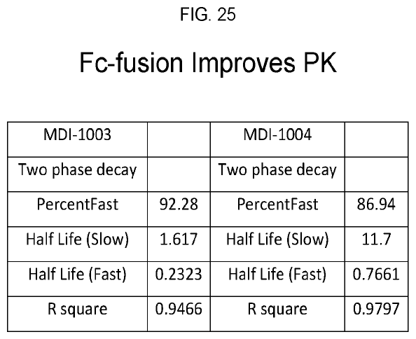

FIG. 25 shows that Fc-fusion improves PK.

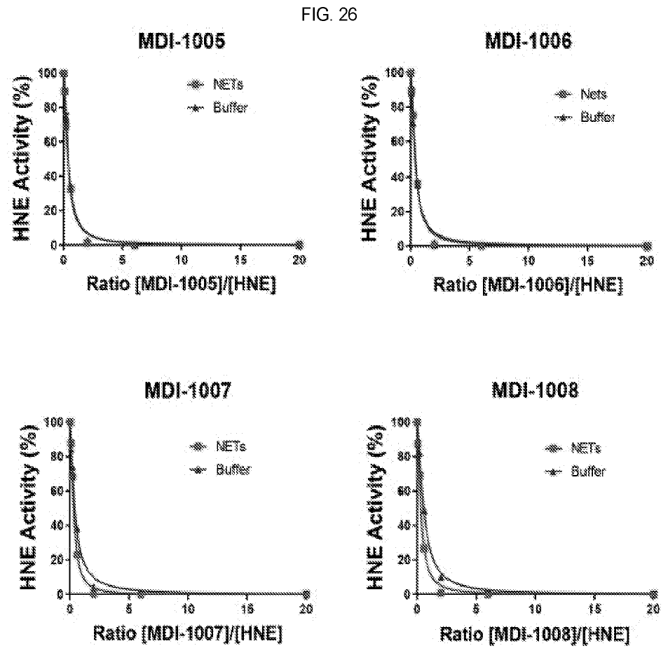

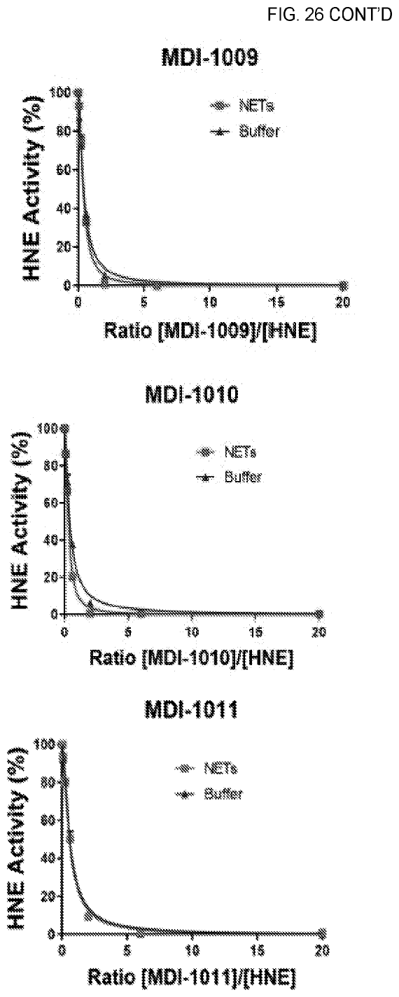

FIG. 26 shows inhibition curves against neutrophil elastase plus or minus DNA NETs. This data indicates that the mutation that provided optimal activity against neutrophil elastase can be combined with each of the mutations shown in Table II that reduce binding to the clearance receptor. The result is improved pharmacokinetics of the molecule. There are 7 total variants, 6 with a single receptor binding mutation and one with 2 receptor binding mutations. The latter double mutant indicates that the mutations can be combined with the potential for even greater reductions in clearance receptor binding. All are of these also contain the I91L, R101A, Q123K, V343A, R346V mutations.

The MDI designation for each variant is:

### K69A is MDI-1005

### K80A is MDI-1006

### K88A is MDI-1007

### K176A is MDI-1008

### K207A is MDI-1009

### K263A is MDI-1010

### K69A-K207A is MDI-1011

These mutations demonstrate reduced binding to the clearance receptor while retaining NET binding and inhibition of neutrophil elastase in the presence of DNA. A preferred mutation is the K207A since this reduces clearance receptor binding of the free inhibitor by 19-fold but only reduces binding of the inhibited protease complex by 1.6-fold (see Table II in the application). This significantly increases the pharmacokinetics of inhibitors with this mutation but still permit the removal of the elastase complex after inhibition.

Having now fully described the invention, it will be understood by those of skill in the art that the same can be performed within a wide and equivalent range of conditions, formulations, and other parameters without affecting the scope of the invention or any embodiment thereof. All patents, patent applications and publications cited herein are fully incorporated by reference herein in their entirety.

### INCORPORATION BY REFERENCE

The entire disclosure of each of the patent documents and scientific articles referred to herein is incorporated by reference for all purposes, including but not limited to any of the references cited herein.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

What is claimed is:

1. A polypeptide comprising a plasminogen-activator inhibitor 1 (PAI-1) variant having one or more of the following mutations within a wild-type human mature PAI-1 amino acid sequence (SEQ ID NO: 3): K69A, K80A, K88A, I91L, R101A, K122A, Q123K, K176A, K207A, K263A, V343A, and R346V.

2. The polypeptide of claim 1, wherein the variant is attached with an Fc domain monomer or moiety.

3. The polypeptide of claim 2, wherein the variant is attached with an Fc domain or moiety.

4. The polypeptide of claim 1, wherein at least one of the mutations are selected from K207A, K88A and K80A.

5. The polypeptide of claim 4, wherein the polypeptide attached with an Fc domain or moiety a) has an amino acid sequence recited in SEQ ID NO: 7, or b) has the following mutations within wild-type human mature PAI-1 amino acid sequence (SEQ ID NO: 3): R101A and Q123K, or c) has the following mutations within wild-type human mature PAI-1 amino acid sequence (SEQ ID NO: 3): K69A, K80A, K88A, I91L, R101A, K122A, Q123K, K176A, K207A, K263A, V343A, and R346V, or d) has the following mutations within wild-type human mature PAI-1 amino acid sequence (SEQ ID NO: 3): I91L, R101A, Q123K, K207A, V343A, R346V.

6. The polypeptide of claim 1, wherein the polypeptide a) has an amino acid sequence recited in SEQ ID NO: 5, or b) has the following mutations within wild-type human mature PAI-1 amino acid sequence (SEQ ID NO: 3): R101A and Q123K, or c) has the following mutations within wild-type human mature PAI-1 amino acid sequence (SEQ ID NO: 3): I91L, R101A, Q123K, V343A, R346V, or d) has the following mutations within wild-type human mature PAI-1 amino acid sequence (SEQ ID NO: 3): I91L, R101A, Q123K, K207A, V343A, R346V.

7. The polypeptide of claim 1, wherein the polypeptide has one or more of the following characteristics a) capable of inhibiting neutrophil elastase (NE) activity, b) diminished ability to bind vitronectin, and c) diminished ability to bind LRP1.

8. The polypeptide of claim 1, wherein the polypeptide is capable of inhibiting neutrophil elastase activity, wherein the neutrophil elastase is bound within a neutrophil extracellular trap (NET).

9. A nucleic acid molecule encoding a polypeptide of claim 1.

10. A vector comprising the nucleic acid molecule of claim 9.

11. A host cell that expresses a polypeptide of claim 1, wherein the host cell comprises a nucleic acid molecule of claim 9 or a vector of claim 10, wherein the nucleic acid molecule or vector is expressed in the host cell.

12. A method of preparing a polypeptide of claim 1, wherein the method comprising: a) providing a host cell comprising a nucleic acid molecule of claim 11 or a vector of claim 10, and b) expressing the nucleic acid molecule or vector in the host cell under conditions that allow for the formation of the polypeptide.

13. A pharmaceutical composition comprising a polypeptide of claim 1, a nucleic acid molecule of claim 9, or a vector of claim 10, and one or more pharmaceutically acceptable carriers or excipients.

14. The pharmaceutical composition of claim 13, wherein the polypeptide is in a therapeutically effective amount.

15. A method of inhibiting NE activity in a subject in need thereof, comprising administering to the subject a therapeutically effective amount of a polypeptide of claim 1, a nucleic acid molecule of claim 9, a vector of claim 10, or a pharmaceutical composition of claim 13.

16. The method of claim 15, wherein the subject has IPF, cystic fibrosis, COPD, ARDS, emphysema, ischemia reperfusion injury, ethanol induced chronic pancreatitis, rheumatoid arthritis (RA), disseminated intravascular coagulation (DIC), ulcerative colitis (UC), Crohn's disease, or dermatological diseases with neutrophil pathology.

17. The method of claim 15, wherein the subject has any disease characterized with aberrant NE activity and/or deficient A1AT activity.

18. A method of treating a subject having IPF, comprising administering to the subject a therapeutically effective amount of a polypeptide of claim 1, a nucleic acid molecule of claim 9, a vector of claim 10, or a pharmaceutical composition of claim 13.

19. A method of treating a subject having cystic fibrosis, comprising administering to the subject a therapeutically effective amount of a polypeptide of claim 1, a nucleic acid molecule of claim 9, a vector of claim 10, or a pharmaceutical composition of claim 13.

20. A method of treating a subject having COPD, comprising administering to the subject a therapeutically effective amount of a polypeptide of claim 1, a nucleic acid molecule of claim 9, a vector of claim 10, or a pharmaceutical composition of claim 13.

21. A method of treating a subject having emphysema, comprising administering to the subject a therapeutically effective amount of a polypeptide of claim 1, a nucleic acid molecule of claim 9, a vector of claim 10, or a pharmaceutical composition of claim 13.

22. A method of treating a subject having ischemia reperfusion injury, comprising administering to the subject a therapeutically effective amount of a polypeptide of claim 1, a nucleic acid molecule of claim 9, a vector of claim 10, or a pharmaceutical composition of claim 13.

23. A method of treating a subject having ethanol induced chronic pancreatitis, comprising administering to the subject a therapeutically effective amount of a polypeptide of claim 1, a nucleic acid molecule of claim 9, a vector of claim 10, or a pharmaceutical composition of claim 13.

24. A method of treating a subject having rheumatoid arthritis (RA), comprising administering to the subject a therapeutically effective amount of a polypeptide of claim 1, a nucleic acid molecule of claim 9, a vector of claim 10, or a pharmaceutical composition of claim 13.

25. A method of treating a subject having disseminated intravascular coagulation (DIC), comprising administering to the subject a therapeutically effective amount of a polypeptide of claim 1, a nucleic acid molecule of claim 9, a vector of claim 10, or a pharmaceutical composition of claim 13.

26. A method of treating a subject having ulcerative colitis (UC), comprising administering to the subject a therapeutically effective amount of a polypeptide of claim 1, a nucleic acid molecule of claim 9, a vector of claim 10, or a pharmaceutical composition of claim 13.

27. A method of treating a subject having Crohn's disease, comprising administering to the subject a therapeutically effective amount of a polypeptide of claim 1, a nucleic acid molecule of claim 9, a vector of claim 10, or a pharmaceutical composition of claim 13.

28. A method of treating a subject having dermatological diseases with neutrophil pathology, administering to the subject a therapeutically effective amount of a polypeptide of claim 1, a nucleic acid molecule of claim 9, a vector of claim 10, or a pharmaceutical composition of claim 13.
